SEQUENCE LISTING (172 sequences)

SEQ ID NO: 1 - protein, 15 aa, Human immunodeficiency virus type 1
EKIRLRPGGKKKYRL

SEQ ID NO: 2 - protein, 15 aa, Human immunodeficiency virus type 1
PGGKKKYRLKHLVWA

SEQ ID NO: 3 - protein, 15 aa, Human immunodeficiency virus type 1
PGGKKKYRMKHLVWA

SEQ ID NO: 4 - protein, 15 aa, Human immunodeficiency virus type 1
KKYRMKHLVWASREL

SEQ ID NO: 5 - protein, 15 aa, Human immunodeficiency virus type 1
KHLVWASRELERFAV

SEQ ID NO: 6 - protein, 15 aa, Human immunodeficiency virus type 1
RELERFAVNPGLLET

SEQ ID NO: 7 - protein, 15 aa, Human immunodeficiency virus type 1
RELERFAVDPGLLET

SEQ ID NO: 8 - protein, 15 aa, Human immunodeficiency virus type 1
SAPKTGTEELRSLYN

SEQ ID NO: 9 - protein, 15 aa, Human immunodeficiency virus type 1
SLYNTVAVLYCVHQR

SEQ ID NO: 10 - protein, 15 aa, Human immunodeficiency virus type 1
SPRTLNAWVKVIEEK

SEQ ID NO: 11 - protein, 15 aa, Human immunodeficiency virus type 1
SPEVIPMFSALSEGA

SEQ ID NO: 12 - protein, 15 aa, Human immunodeficiency virus type 1
ATPQDLNMMLNIVGG

SEQ ID NO: 13 - protein, 15 aa, Human immunodeficiency virus type 1
ATPQDLNTMLNIVGG

SEQ ID NO: 14 - protein, 15 aa, Human immunodeficiency virus type 1
QDLNMMLNIVGGHQA

SEQ ID NO: 15 - protein, 15 aa, Human immunodeficiency virus type 1
MMLNIVGGHQAAMQM

SEQ ID NO: 16 - protein, 15 aa, Human immunodeficiency virus type 1
SNPPIPVGDIYKRWI

SEQ ID NO: 17 - protein, 15 aa, Human immunodeficiency virus type 1
KRWIILGLNKIVRMY

SEQ ID NO: 18 - protein, 15 aa, Human immunodeficiency virus type 1
LGLNKIVRMYSPVSI

SEQ ID NO: 19 - protein, 15 aa, Human immunodeficiency virus type 1
LGLNKIVRMYSPTSI

SEQ ID NO: 20 - protein, 15 aa, Human immunodeficiency virus type 1
RMYSPVSILDIKQGP

SEQ ID NO: 21 - protein, 16 aa, Human cytomegalovirus
NSSRHSGKCRRQRRAL

SEQ ID NO: 22 - protein, 15 aa, Human cytomegalovirus
ALSAPPLTFLATTTT

SEQ ID NO: 23 - protein, 15 aa, Human cytomegalovirus
RQPRVHRGTYHLIQL

SEQ ID NO: 24 - protein, 16 aa, Human cytomegalovirus
TYHLIQLHLDLRPEEL

SEQ ID NO: 25 - protein, 15 aa, Human cytomegalovirus
RPEELRDPFQILLST

SEQ ID NO: 26 - protein, 15 aa, Human cytomegalovirus
SDVRPAFSLFPARPG

SEQ ID NO: 27 - protein, 15 aa, Human cytomegalovirus
FPARPGCHILRSVID

SEQ ID NO: 28 - protein, 15 aa, Human cytomegalovirus
FALRIITPPLKRVPL

SEQ ID NO: 29 - protein, 15 aa, Human cytomegalovirus
FALRIITPPLKRVPL

SEQ ID NO: 30 - protein, 15 aa, Human cytomegalovirus
TDADLTPTLTVRVRH

SEQ ID NO: 31 - protein, 15 aa, Human cytomegalovirus
ISGPRGLTSRISARL

SEQ ID NO: 32 - protein, 16 aa, Human cytomegalovirus
DPNESPPDLTLSSLTL

SEQ ID NO: 33 - protein, 15 aa, Human cytomegalovirus
TLYQDGMLRFNVTCD

SEQ ID NO: 34 - protein, 15 aa, Human cytomegalovirus
TEAPADPVAFRLRLR

SEQ ID NO: 35 - protein, 15 aa, Human cytomegalovirus
FRLRLRRETVRRPFF

SEQ ID NO: 36 - protein, 15 aa, Human cytomegalovirus
RRPFFSDAPLPYFVP

SEQ ID NO: 37 - protein, 15 aa, Human cytomegalovirus
DEGLEVRVPYELTLK

SEQ ID NO: 38 - protein, 15 aa, Human cytomegalovirus
RIYRRFYGPYLGVFV

SEQ ID NO: 39 - protein, 15 aa, Human cytomegalovirus
YLGVFVPHNRQGLKM

SEQ ID NO: 40 - protein, 15 aa, Human cytomegalovirus
LKMPVTVWLPRSWLE

SEQ ID NO: 41 - protein, 15 aa, Human cytomegalovirus
TVWLPRSWLELTVLV

SEQ ID NO: 42 - protein, 15 aa, Human cytomegalovirus
ATFPRDALLGRLYFI

SEQ ID NO: 43 - protein, 15 aa, Human cytomegalovirus
ALLGRLYFISSKHTL

SEQ ID NO: 44 - protein, 15 aa, Human coronavirus
NSVLLFLAFVVFLLV

SEQ ID NO: 45 - protein, 15 aa, Human coronavirus
LLFLAFVVFLLVTLA

SEQ ID NO: 46 - protein, 15 aa, Human coronavirus
LAWIMLLQFAYSNRN

SEQ ID NO: 47 - protein, 15 aa, Human coronavirus
MESELVIGAVIIRGH

SEQ ID NO: 48 - protein, 15 aa, Human coronavirus
MACIVGLMWLSYFVA

SEQ ID NO: 49 - protein, 15 aa, Human coronavirus
PLMESELVIGAVIIR

SEQ ID NO: 50 - protein, 15 aa, Human coronavirus
GKMKELSPRWYFYYL

SEQ ID NO: 51 - protein, 15 aa, Human coronavirus
QQQQGQTVTKKSAAE

SEQ ID NO: 52 - protein, 15 aa, Human coronavirus
KMKELSPRWYFYYLG

SEQ ID NO: 53 - protein, 15 aa, Human coronavirus
ETALALLLLDRLNQL

SEQ ID NO: 54 - protein, 15 aa, Human coronavirus
THTMIFDNAFNCTFE

SEQ ID NO: 55 - protein, 15 aa, Human coronavirus
CSVKSFEIDKGIYQT

SEQ ID NO: 56 - protein, 15 aa, Human coronavirus
NCVADYSVLYNSTFF

SEQ ID NO: 57 - protein, 15 aa, Human coronavirus
IAPGQTGVIADYNYK

SEQ ID NO: 58 - protein, 15 aa, Human coronavirus
VSLLRSTSQKSIVAY

SEQ ID NO: 59 - protein, 15 aa, Human coronavirus
SITTEVMPVSMAKTS

SEQ ID NO: 60 - protein, 15 aa, Human coronavirus
QVKQMYKTPTLKYFG

SEQ ID NO: 61 - protein, 15 aa, Human coronavirus
QIPFAMQMAYRFNGI

SEQ ID NO: 62 - protein, 15 aa, Human coronavirus
ISSVLNDILSRLDKV

SEQ ID NO: 63 - protein, 15 aa, Human coronavirus
RLDKVEAEVQIDRLI

SEQ ID NO: 64 - protein, 15 aa, Human coronavirus
YFVGYLKPTTFMLKY

SEQ ID NO: 65 - protein, 15 aa, Human coronavirus
SNCVADYSVLYNSTF

SEQ ID NO: 66 - protein, 15 aa, Human coronavirus
RDPKTSEILDISPCS

SEQ ID NO: 67 - protein, 15 aa, Human coronavirus
GSFCTQLNRALSGIA

SEQ ID NO: 68 - protein, 15 aa, Human coronavirus
VKQMYKTPTLKYFGG

SEQ ID NO: 69 - protein, 15 aa, Human coronavirus
AGFMKQYGECLGDIN

SEQ ID NO: 70 - protein, 15 aa, Human coronavirus
PPLLTDDMIAAYTAA

SEQ ID NO: 71 - protein, 15 aa, Human coronavirus
ISSVLNDILSRLDKV

SEQ ID NO: 72 - protein, 15 aa, Human coronavirus
TQQLIRAAEIRASAN

SEQ ID NO: 73 - protein, 15 aa, Human coronavirus
LDSFKEELDKYFKNH

SEQ ID NO: 74 - protein, 15 aa, Bacillus anthracis
ESESSSQGLLGYYFS

SEQ ID NO: 75 - protein, 15 aa, Bacillus anthracis
NIPSENQYFQSAIWS

SEQ ID NO: 76 - protein, 15 aa, Bacillus anthracis
QYQRENPTEKGLDFK

SEQ ID NO: 77 - protein, 15 aa, Bacillus anthracis
ISNIHEKKGLTKYKS

SEQ ID NO: 78 - protein, 15 aa, Bacillus anthracis
VSPEARHPLVAAYPI

SEQ ID NO: 79 - protein, 15 aa, Bacillus anthracis
PIYNVLPTTSLVLGK

SEQ ID NO: 80 - protein, 15 aa, Bacillus anthracis
KAKENQLSQILAPNN

SEQ ID NO: 81 - protein, 15 aa, Bacillus anthracis
QVYGNIATYNFENGR

SEQ ID NO: 82 - protein, 15 aa, Bacillus anthracis
NIDKDIRKILSGYIV

SEQ ID NO: 83 - protein, 15 aa, Bacillus anthracis
FIDFKKYNDKLPLYI

SEQ ID NO: 84 - protein, 15 aa, Variola virus
SKNEAEKLITSAFDL

SEQ ID NO: 85 - protein, 15 aa, Variola virus
RQSSCKMALLFKNLA

SEQ ID NO: 86 - protein, 15 aa, Variola virus
ICLKDKSELYSAYKT

SEQ ID NO: 87 - protein, 15 aa, Variola virus
TRERNKIVELEKELN

SEQ ID NO: 88 - protein, 15 aa, Variola virus
ICIRISMVISLLSMI

SEQ ID NO: 89 - protein, 15 aa, Variola virus
GLYYQGSCYIFHSDY

SEQ ID NO: 90 - protein, 15 aa, Variola virus
TLPNKSDVLTTWLID

SEQ ID NO: 91 - protein, 15 aa, Variola virus
GHSETDTTTEYLLPN

SEQ ID NO: 92 - protein, 15 aa, Variola virus
GPSTYYPHYKSCALV

SEQ ID NO: 93 - protein, 15 aa, Variola virus
TSPARENYFMRWLSD

SEQ ID NO: 94 - protein, 15 aa, Variola virus
REVNKALYDLQRSAM

SEQ ID NO: 95 - protein, 15 aa, Variola virus
VIPAKKIIDWKNANP

SEQ ID NO: 96 - protein, 15 aa, Variola virus
MASLLYFILFLLFVC

SEQ ID NO: 97 - protein, 15 aa, Variola virus
IFTDASTVASAQIYL

SEQ ID NO: 98 - protein, 15 aa, Variola virus
TSHLNPSIEKHVGIY

SEQ ID NO: 99 - protein, 15 aa, Variola virus
LSAKVYMLENIQVMK

SEQ ID NO: 100 - protein, 15 aa, Variola virus
TYHLENDKIEDLINQ

SEQ ID NO: 101 - protein, 15 aa, Variola virus
DSMYVIPDELIDVLK

SEQ ID NO: 102 - protein, 15 aa, Variola virus
AYVPAMFTAALNIQT

SEQ ID NO: 103 - protein, 15 aa, Variola virus
VIGVIILAALFMYYA

SEQ ID NO: 104 - protein, 9 aa, Homo sapiens
KGEEELQKY

SEQ ID NO: 105 - protein, 9 aa, Homo sapiens
FTEIQKLVL

SEQ ID NO: 106 - protein, 9 aa, Homo sapiens
FTESRTLHR

SEQ ID NO: 107 - protein, 9 aa, Homo sapiens
SSLVVDETY

SEQ ID NO: 108 - protein, 9 aa, Homo sapiens
ITECCKLTT

SEQ ID NO: 109 - protein, 9 aa, Homo sapiens
FSDDKFIFH

SEQ ID NO: 110 - protein, 9 aa, Homo sapiens
ETFMNKFIY

SEQ ID NO: 111 - protein, 9 aa, Homo sapiens
SSELMAITR

SEQ ID NO: 112 - protein, 9 aa, Homo sapiens
AEEGQKLIS

SEQ ID NO: 113 - protein, 9 aa, Homo sapiens
WVESIFLIF

SEQ ID NO: 114 - protein, 9 aa, Homo sapiens
IADFSGLLE

SEQ ID NO: 115 - protein, 9 aa, Homo sapiens
SRTLHRNEY

SEQ ID NO: 116 - protein, 9 aa, Homo sapiens
AQFVQEATY

SEQ ID NO: 117 - protein, 9 aa, Homo sapiens
FLASFVHEY

SEQ ID NO: 118 - protein, 9 aa, Homo sapiens
VILRVAKGY

SEQ ID NO: 119 - protein, 9 aa, Homo sapiens
IQESQALAK

SEQ ID NO: 120 - protein, 9 aa, Homo sapiens
ITEEQLEAV

SEQ ID NO: 121 - protein, 9 aa, Homo sapiens
HEKEILEKY

SEQ ID NO: 122 - protein, 9 aa, Homo sapiens
QSEEGRHNC

SEQ ID NO: 123 - protein, 9 aa, Homo sapiens
LSEPAELTD

SEQ ID NO: 124 - protein, 9 aa, Homo sapiens
GSEPCALPE

SEQ ID NO: 125 - protein, 9 aa, Homo sapiens
SHSFPHPLY

SEQ ID NO: 126 - protein, 9 aa, Homo sapiens
SPECIFICA

SEQ ID NO: 127 - protein, 9 aa, Homo sapiens
ASESEMINI

SEQ ID NO: 128 - protein, 9 aa, Homo sapiens
ESEMININP

SEQ ID NO: 129 - protein, 9 aa, Homo sapiens
LTDAVKVMD

SEQ ID NO: 130 - protein, 9 aa, Homo sapiens
ALPERPSLY

SEQ ID NO: 131 - protein, 9 aa, Homo sapiens
SLYTKVVHY

SEQ ID NO: 132 - protein, 9 aa, Homo sapiens
ATESPECIF

SEQ ID NO: 133 - protein, 9 aa, Homo sapiens
RTEINKALL

SEQ ID NO: 134 - protein, 9 aa, Homo sapiens
EMENGELAS

SEQ ID NO: 135 - protein, 9 aa, Homo sapiens
LYDMSLLKN

SEQ ID NO: 136 - protein, 9 aa, Homo sapiens
SHDLMLLRL

SEQ ID NO: 137 - protein, 9 aa, Homo sapiens
SIEPEEFLT

SEQ ID NO: 138 - protein, 9 aa, Homo sapiens
RTEINSEME

SEQ ID NO: 139 - protein, 9 aa, Homo sapiens
EPALGTTCY

SEQ ID NO: 140 - protein, 9 aa, Homo sapiens
SGDSGGPLV

SEQ ID NO: 141 - protein, 9 aa, Homo sapiens
LPERPSLYT

SEQ ID NO: 142 - protein, 9 aa, Homo sapiens
ASEMINPRT

SEQ ID NO: 143 - protein, 9 aa, Homo sapiens
EVDPIGHLY

SEQ ID NO: 144 - protein, 9 aa, Homo sapiens
SSLPTTMNY

SEQ ID NO: 145 - protein, 9 aa, Homo sapiens
ISGGPHISY

SEQ ID NO: 146 - protein, 9 aa, Homo sapiens
LVHFLLLKY

SEQ ID NO: 147 - protein, 9 aa, Homo sapiens
FATCLGLSY

SEQ ID NO: 148 - protein, 9 aa, Homo sapiens
GSVVGNWQY

SEQ ID NO: 149 - protein, 9 aa, Homo sapiens
LGDPKKLLT

SEQ ID NO: 150 - protein, 9 aa, Homo sapiens
FVQENYLEY

SEQ ID NO: 151 - protein, 9 aa, Homo sapiens
GENFAMILY

SEQ ID NO: 152 - protein, 9 aa, Homo sapiens
ATEEQQAAS

SEQ ID NO: 153 - protein, 9 aa, Homo sapiens
ATEDANTIG

SEQ ID NO: 154 - protein, 9 aa, Homo sapiens
RGEALGLVG

SEQ ID NO: 155 - protein, 9 aa, Homo sapiens
ESEFQAALS

SEQ ID NO: 156 - protein, 9 aa, Homo sapiens
GSDPACYEF

SEQ ID NO: 157 - protein, 9 aa, Homo sapiens
SPDPPQSPQ

SEQ ID NO: 158 - protein, 9 aa, Homo sapiens
GIEMCAANY

SEQ ID NO: 159 - protein, 9 aa, Homo sapiens
AQDAPPLPV

SEQ ID NO: 160 - protein, 9 aa, Homo sapiens
AADHRQLQL

SEQ ID NO: 161 - protein, 9 aa, Homo sapiens
RTEINHMSA

SEQ ID NO: 162 - protein, 9 aa, Homo sapiens
GPESRLLEF

SEQ ID NO: 163 - protein, 9 aa, Homo sapiens
FTVSGNILT

SEQ ID NO: 164 - protein, 9 aa, Homo sapiens
PESRLLEFY

SEQ ID NO: 165 - protein, 9 aa, Homo sapiens
LPVPGVLLK

SEQ ID NO: 166 - protein, 9 aa, Homo sapiens
CFLPVFLAQ

SEQ ID NO: 167 - protein, 9 aa, Homo sapiens
SSCLQQLSL

SEQ ID NO: 168 - protein, 9 aa, Homo sapiens
DADGPGGPG

SEQ ID NO: 169 - protein, 9 aa, Homo sapiens
IPDGPGGNA

SEQ ID NO: 170 - protein, 9 aa, Homo sapiens
LLEFYLAMP

SEQ ID NO: 171 - protein, 9 aa, Homo sapiens
NYESPRTEI

SEQ ID NO: 172 - protein, 9 aa, Homo sapiens
QAEGRGTGG

(19) United States
(12) Patent Application Publication
(10) Pub. No.: US 2007/0178533 A1
(43) Pub. Date: Aug. 2, 2007

(54) Method and diagnostic tests based on flow cytometric analysis of antigen-specific t lymphocytes

(76) Inventors: Fabrizio Poccia, Roma (IT); Christiana Gioia, Roma (IT); Chiara Agrati, Roma (IT); Carla Montesano, Roma (IT); Massimo Amicosante, Roma (IT); Rita Casetti, Cave (IT); Gianpiero D'Offizi, Roma (IT); Douglas Horejsh, Ciampino (IT); Federico Martini, Roma (IT); Maria Rosaria Capobianchi, Roma (IT); Leopoldo Paolo Pucillo, Roma (IT); Raffaele Perrone Donnorso, Roma (IT); Giuseppe Ippolito, Roma (IT)

Correspondence Address: STETINA BRUNDA GARRED & BRUCKER, 75 ENTERPRISE, SUITE 250, ALISO VIEJO, CA, 92656, US

(21) Appl. No.: 10/567,541

(22) PCT Filed: Aug. 5, 2004
(86) PCT No.: PCT/EP04/51726

§ 371 Date: Feb. 6, 2006

(30) Foreign Application Priority Data
Aug. 5, 2003 (IT) RM2003A000386

(51) Int. Cl. G01N 33/567

(52) U.S. Cl. 435/7.2

(57) ABSTRACT

The present invention provides a method for the immuno-diagnosis of diseases with different aetiology (infectious diseases, tumors etc) by measurement of the T cell response J, B and NK lymphocytes) induced by a set of diseasespecific antigens. The method is based on the quantitative determination of antigenspecific T lymphocytes (referred as Ag-Sp), stimulated by using a newly devised pathology-specific antigen or epitope compositions which represent further embodiments of the invention. After stimulation, the selective measurement of the Ag-Sp T lymphocytes is performed by: A) monoclonal antibodies recognizing membrane structures of T lymphocytes and of their sub-populations; B) monoclonal antibodies binding to cytokines accumulating at intracellular level after the stimulation with the antigen; or C) mixtures of A) and B). The flow cytometric detection of the presence of markers of differentiation on T lymphocytes and of intracytoplasmic cytokines allows the acquisition of both qualitative and quantitative results. The invention also provides diagnostic kits for performing the method of the invention.

### FIELD OF THE INVENTION

The present invention refers to a method and corresponding diagnostic tests to assay immune responses to antigens associated with pathologies that generate T cell responses. The test is based on the flow-cytometry analysis of the antigen-specific T lymphocytes (referred as Ag-Sp T lymphocytes).

More particularly the invention refers to a method and corresponding diagnostic tests to simultaneously assay the exposure to antigens associated with biological threat agents. The method can also be applied to other clusters of disease and may allow to determine at once the occurrence of respiratory infections, sexually transmitted diseases, in utero infections, post-transplant infection, blood borne infections or neoplastic diseases with known tumor associated antigens.

### BACKGROUND

Although the last natural case of smallpox was reported in Somalia in 1977, this orthopoxvirus remains a source of concern. No evidence exists that smallpox will recur as an endemic disease, but the virus may have been acquired for use in biological warfare or bioterrorist attacks. Assuming an average of 15 days needed for infected persons to become infectious, delay in intervention will be costly, increasing the total number of cases. Furthermore, the recent outbreak of the severe acute respiratory syndrome coronavirus and the first documented outbreak of monkeypoxvirus in the Western Hemisphere underline the ever-present risk of epidemic extension of zoonosis and raise concerns about the medical and social effect of reemerging orthopoxvirus infection in humans. Moreover, the intentional spread of envelopes containing Anthrax spore in the Unites States has determined a great alarm and some deaths. The diagnosis of lung Anthrax infection has a differential diagnosis from plague and other common infections such as bacterial pulmonitis and influenza infection. During the epidemic spread of a biological threat agent, evaluating exposed persons and containing the infected population should be the first priorities. A local outbreak of a biological threat agent would require rapid and sensitive diagnostics, including novel assays based on host responses.

Commonly used tests for the immuno-diagnosis of diseases are based on the determination of serum antigens or specific antibodies produced by B-lymphocytes (Uhr J W, Finkelstein M S. The kinetics of antibody formation. Prog Allergy. 1967;10:37-83). However, these tests based on B lymphocytes fail to give positive results until two weeks post-antigen exposure, allowing for the minimal time necessary to activate the B lymphocytes. In some cases, months are necessary to generate a significant result.

In vitro tests have been recently developed for the measurement of cell-mediated immunity that develops 7-10 days after antigen exposure. These methods may be used to analyse the presence of antigen-specific cells, need some days to be performed and are preferentially based on ELISA tests (e.g. patent application WO02059605) or on cellular proliferation tests (e.g. WO0011476). Both of these approaches give only qualitative results and provide information about the ability of immune cell to recognize the antigen although they do not allow the measurement of the frequency of the cells responding to the antigen stimulation. Methods have also been published to estimate on whole blood the presence of M. tuberculosis specific cells (e.g. patents WO02/059605 and WO87/05400). However, these approaches were restricted to the M.tuberculosis-specific response, required a long stimulation time and were poorly sensitive. The possibility to monitor the frequency of T cells producing intracellular cytokines by flow cytometry is faster and sensitive (Betts M R, Casazza J P, Koup R A. Monitoring HIV-specific CD8 T cell responses by intracellular cytokine production. Immunology Letters 2001; 79:117-125; Elkington R, Walker S, Crough T, Menzies M, Tellam J, Bharadwaj M, Khanna R. Ex vivo profiling of CD8+−T-cell responses to human cytomegalovirus reveals broad and multispecific reactivities in healthy virus carriers. J Virol. 2003 May; 77(9):5226-40.) and a computer-based method to indentify the common antigens to monitor HIV infection was recently described (Amicosante M, Gioia C, Montesano C, Casefti R, Topino S, D'Offizi G, Cappelli G, Ippolito G, Colizzi V, Poccia F, Pucillo L P. Computer-based design of an HLA-haplotype and HIV-clade independent cytotoxic T-lymphocyte assay for monitoring HIV-specific immunity. Mol Med. 2002;8:798-807). However, these methods do not allow to discriminate pathogenic from nonpathogenic species of the same family of microorganism which is crucial for the detection of biological threat agents (for example to distinguish the highly pathogenic variola virus from the safe administration of a vaccine or to discriminate the dangerous SARS coronavirus from the common cold OC43 or E229 coronavirus).

On the contrary the method disclosed in the present invention:
allows the fast, powerful and specific identification of persons exposed to bio-terrorism threat agents discriminating between pathogenic and nonpathogenic strains;
allows the discrimination between a memory response to a vaccine from the primary response to a dangerous pathogen, in this way identifying persons which are vaccinated for a given agent from non-immune exposed persons;
provides both qualitative and quantitative results, expressed either by frequency or by absolute values of antigen-specific T lymphocytes present in the peripheral blood;
allows the characterization of the T cell subset or the effector stage known to respond to a specific Patho-tope (pathology-specific epitopes) (eg. CD4 or CD8 T cells, CD45-RA and CD27, etc.), resulting in a more specific and sensitive identification of the T cell response for diagnostic purpose and minimizing the aspecific background of the diagnostic test;
provides specific arrays comprising a panel of pathology-specific epitopes (Patho-topes) covering many different set of pathologies in a multiplex application. Such arrays are assembled and manufactured as a ready-to-use pathology-specific arrays which can be assembled in ready to use diagnostic kits that can be performed in less than 24 hours, sometimes in less than 8 hours, and are effective also using cryopreserved samples.

### SUMMARY OF THE INVENTION

The present invention provides a method for the set-up of a specific kit allowing the immune diagnosis, in particular of bio-terrorism agent exposure by measuring the immune response to antigens associated with all those pathologies that generate a T cell response. Specifically, the method is focused on the use of pathogen-discriminating epitopes that have been selected between commercially available recombinant proteins, and designed as a set of synthetic peptides (peptide composition) efficiently and, in general, promiscuously inducing the stimulation of T-lymphocytes specific for pathogenic and nonpathogenic variants of the biological threat agent. The quantitative determination of antigen-specific T lymphocytes (referred as Ag-Sp), was analysed by using these newly devised pathology-specific antigen or epitope compositions which represent a further embodiment of the invention (Patho-tope arrays). After the stimulation, the test uses a rapid method for the selective measurement of the Ag-Sp T lymphocytes that are identified through: A) monoclonal antibodies recognizing membrane structures of T lymphocytes and of their sub-populations; B) monoclonal antibodies binding to cytokines accumulating at intracellular level after the stimulation with the antigen; and C) mixtures of A) and B). The flow cytometric detection of the presence of markers of differentiation on T lymphocytes and of intracytoplasmic cytokines allows the acquisition of both qualitative and quantitative results. The diagnostic test described in the present invention is performed using venous blood, is composed by a simple reagent kit, and uses a flow cytometer for read-out—commonly used in laboratories of clinical pathology for the quantification of the T, B and NK lymphocytes. The availability of mobile flow-cytometer units may allow the use of this assay under field investigation conditions.

Additional embodiments will become evident from the following detailed description of the invention.

### BRIEF DESCRIPTION OF THE FIGURES

FIG. 1 schematically shows the test of immune diagnosis though the quantitative analysis of the Ag-Sp T lymphocytes.

FIG. 2 shows T cell response profiling by the use of different pathology-specific epitopes focusing on conventional and biological threat agents.

FIG. 3 shows the T cell response to coronavirus proteins and selected peptides.

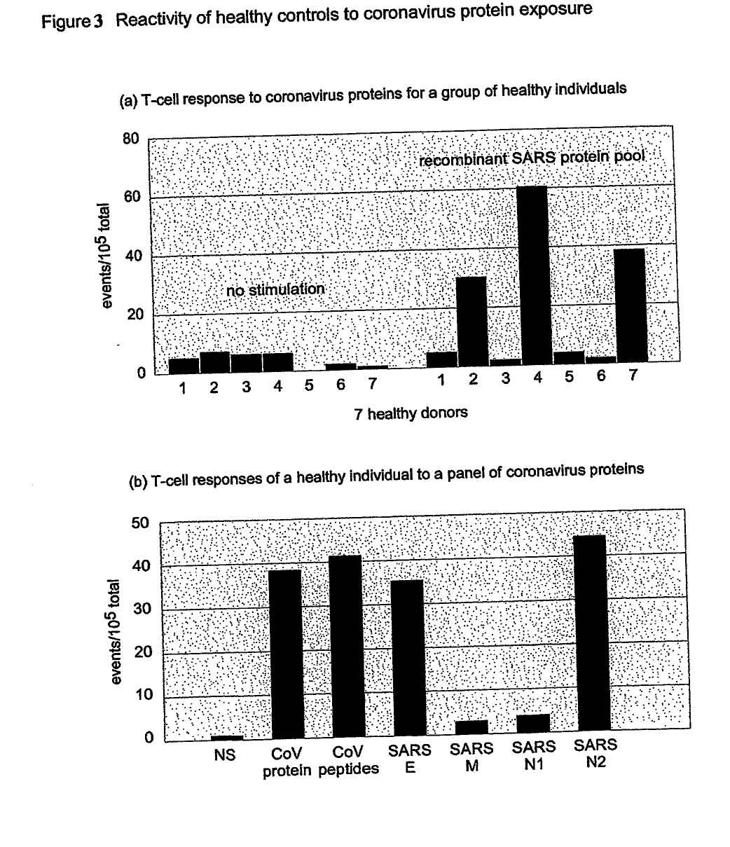

FIG. 4 describes the method of selection of pathogen-discriminating peptides (peptide composition) promiscuously inducing the stimulation of T-lymphocytes specific for pathogenic and nonpathogenic variants of the biological threat agent.

### DETAILED DESCRIPTION OF THE INVENTION

The present invention refers to an immune diagnostic assay based on the stimulation of T lymphocytes by a panel of pathology-specific antigens in the form of: lysates, epitopes defined by synthetic peptides or purified proteins either recombinant or natural (Patho-tope arrays) which allows the quantification and the characterization of antigen-specific T lymphocytes immunity. Specific T-lymphocytes appear only 7-10 days after antigen exposure and are therefore detectable before specific antibodies are produced. The test can be performed on human or animal venous blood samples.

The method for in vitro immuno-diagnosis of antigen-specific (Ag-Sp) T lymphocytes is based on the preparation of compositions able to stimulate the T lymphocytes; such compositions (also called Ag-Sp or Patho-tope arrays or, simply, preparations or stimuli) comprise at least one among the antigens in different forms selected in the group of: (a) raw protein extract, (b) purified or recombinant proteins, (c) synthetic peptides and combinations of (a), (b) and (c).

In particular, when such stimuli are based on antigens originating from pathogens they will be identified as “pathogen-specific” and when based on antigens originating from strains used for making vaccines they will be identified as “vaccine-specific”. Accordingly, the method of the invention comprises the following steps:
i) isolation of peripheral blood mononuclear cells (PBMC) from a sample of human or animal venous blood;
ii) preparation of at least one, preferably both of the following samples: a panel of pathogen-specific stimuli, comprising the above mentioned components (a), (b), (c) and combinations thereof carrying antigens present only in pathogens; a panel of vaccine-specific stimuli, comprising the above mentioned components (a), (b), (c) and combinations thereof carrying antigens present only in strains used for vaccine preparations;
iii) preparation of a negative control comprising cells cultivated in vitro in complete medium without stimuli (this negative control makes it possible to evaluate the aspecific background response); and a positive control comprising cells cultivated in vitro in complete medium with an aspecific stimulus such as a pharmacologically-induced one, e.g. phorbol myristic acetate and ionomycin, (this positive control allows to evaluate the viability and the response capability of responder cells);
iv) stimulation of said T-lymphocytes with the vaccine-specific or the a pathogen-specific preparations (or stimuli) in the presence of a costimulus such as an anti-CD28 and/or an anti-CD49d monoclonal antibody;
v) incubation;
vi) selective staining by immunofluorescence;
vii) flow-cytometry acquisition and analysis;
viii) measurement and characterization of the immune response.

Data evaluation and response are given by identifying a cut-off value for the specific response, set by common statistical methods as the average plus two times the standard deviation of the T cell response frequency obtained from a sample of healthy persons, such as blood donors (the identification of this value allows to discriminate between healthy uninfected persons and infected or vaccinated persons).

Only as an indication of the kind of evaluations that can be obtained by the present method, the following conclusions can be made:
normal healthy persons have a frequency of responding pathogen-specific T cells below the cut-off threshold,
infected persons have a frequency of responding pathogen-specific T cells above the cut-off threshold;
chronically infected and acutely infected persons are discriminated by longitudinal follow-up:
   cronically infected persons, when studied over a period of three months, will show a steady level of the frequency of responding pathogen-specific T cells;
   acutely infected persons, when studied over a period of three months, will show a reduction of the frequency of responding pathogen-specific T cells.
   vaccinated persons have a frequency of responding vaccine-specific T cells above the cut-off threshold and a frequency of responding pathogen-specific T cells below the cut-off threshold.

The method and corresponding data evaluation can be computer-made and the results can be generated by means of a program comprising software paths that carry out the above mentioned steps.

The pathogen specific preparation according to point ii) is designed for the specific pathology under examination, and represents further embodiments of the invention.

Antigens in different forms (mixed compositions) can be combined for better efficiency in lymphocytes stimulation and detection.

According to a further aspect of the invention, mixtures (or compositions) of synthetic peptides have been designed, selected and validated to provide set of synthetic peptides (peptide composition) efficiently and, in general, promiscuously inducing stimulation of specific T-lymphocytes and their detection according to the method of the invention.

According to a further embodiment, the invention is related to specific combinations of antigenic peptides referred to as compositions, designed, tested and validated to detect with high sensitivity and specificity pathologies or group of pathologies, for which specific T-lymphocytes are usually produced in vivo. Peptide sequences and combination of compositions are reported ahead and in the sequence listing. Peptides and peptide mixes are designed according to the following procedure, summarized for HLA-Class I (CD8) peptides:
1) a protein relevant for the pathogen or pathological status under scrutiny (a protein relevant in that used or possibly usable to make a vaccine being comprised within the above definition) is chosen, whose expression could be indicative of vaccination against the pathogen or of the pathogen or pathological status presence. The proteins of interest are selected with different criteria for viruses, bacteria or tumors. For viruses, proteins are selected from the core, from the surface/envelope and from regulatory proteins focusing on more variable among related strains with a different pathogenicity. For bacteria, proteins are selected among toxins that are associated to pathogenicity. For tumors, proteins are selected from known tumor associated antigens. As an example, according to the present invention pathogens may be selected in the following non exhaustive group: Variola (Ortho-Poxviruses), Anthrax (B.anthracis), Plague (Yersinia pestis), Tularemia (Francisella tularensis) and SARS (Coronavirus). For Ortho-Poxviruses the following proteins are preferably selected: Protein A10L, Protein A27, Protein 33R, Protein C7L, Protein D8L, Protein E3L, Protein H3L, Protein H6R, Protein K1L, Protein M1R. For Anthrax (B.anthracis): Protective antigen Protein peptides are preferred. For Plague (Yersinia pestis) the Capsular F1 antigen is preferred. For Tularemia (Francisella tularensis) the LPS antigen is preferred. For coronavirus (SARS) the following are preferred: SARS coronavirus protein E, the SARS coronavirus protein M, the SARS coronavirus protein N, the SARS coronavirus protein S, the SARS coronavirus Nucleocapsid protein.
2) if necessary, a “consensus sequence” is built, accounting for any possible strain or dade or subtype heterogeneity. Specifically, the different sequences available on the databank are compared with a specific software (ClustalW, http://www.ebi.ac.uk/clustalw/) that produces the consensus sequence on the basis of best match for the selected sequences. The consensus sequence is necessary when for the pathogen under scrutiny, a dade or subgroup heterogeneity is expected; the peptides built on the consensus sequence will work on any dade or subgroup of the pathogen, in this way limiting the possibility of false negative results. In the illustrative examples herewith shown there was no need to build a consensus sequence as there were no heterogeneity or variants;
3) for each protein or for each consensus sequence, a listing of all the possible HLA Class I-binding peptides is built, complete with binding scores, by using HLA-binding prediction software on the net (e.g. SYFPEITHY (http://syfpeithi.bmi-heidelberg.com/) or BIMAS (http://bimas.dcrt.nih.gov/molbio/hla_bind/));
4) among the peptides, those ranking higher than 24 in the SYFPEITHY program predictions and peptides that scored greater than 100 from the BIMAS program predictions are chosen;
5) the individual top binding score peptides are aligned on the protein sequence, in order to identify immunodominant regions, and peptides which bind to at a minimum of two different HLA loci (HLA-A and -B, or HLA-A and -C, or HLA-B and -C), or better, to all three loci (HLA-A and -B and -C) are selected;
6) for each region, several peptides (ranging from 9-mers to 20-mers) are designed to overlap and include each immunodominant region;
7) the individual peptides are tested for specificity for the microbial variant by using protein-protein BLAST (http://www.ncbi.nlm.nih.gov/blast/Blast.cgi) in order to exclude non-specific sequences. According to a preferred embodiment the research if performed in a pairwise manner on the whole database including all non-redundant GenBank CDS translations plus PDB plus SwissProt plus PIR plus PRF but excluding environmental samples sequences;
8) a (peptide mixture or a) composition is designed, comprising individual peptides or group of peptides for each antigen in a way that covers a relevant fraction (greater than 90%) of all the possible HLA haplotypes in the selected population.

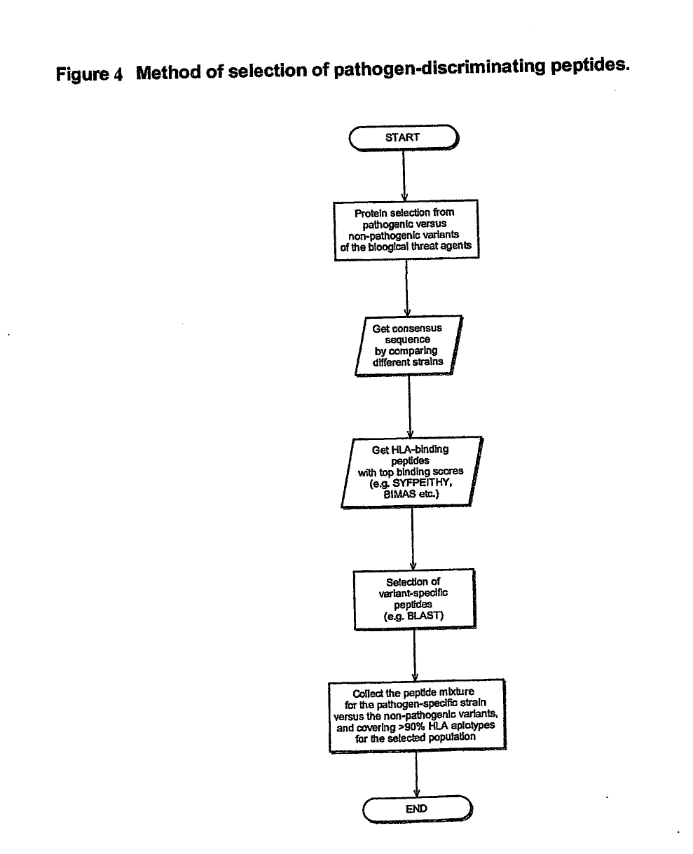

The method to generate the peptides as described in the above and schematically illustrated in FIG. 4, can be computer-made and the results can be generated by means of a program comprising software paths that carry out the above mentioned steps.

According to the method of the present invention, particularly preferred compositions have been devised, comprising at least one, preferably two, more preferably more than two of the following peptides selected in the group of:

Ortho-Poxviruses,
Protein A10L peptides (from sequence ID84 to 85 of this application)
Protein A27 peptides (from sequence ID86 to 87 of application)
Protein A33R peptides (from sequence ID88 to 90 of this application)
Protein C7L peptides (from sequence ID91 to 92 of this application)
Protein D8L peptides (from sequence ID93 of this application)
Protein E3L peptides (from sequence ID94 to 95 of this application)
Protein H3L peptides (from sequence ID96 to 97 of this application)
Protein H6R peptides (from sequence ID98 to 99 of this application)
Protein K1L peptides (from sequence ID100 to 101 of this application)
Protein M1R peptides (from sequence ID102 to 103 of this application)

Anthrax (B.anthracis):
Protective antigen Protein peptides (from sequence ID74 to 83 of this application);

SARS coronavirus composition. It comprises the following antigen preparations:
SARS coronavirus protein E peptides (sequence ID44 and 59 of this application)
SARS coronavirus protein M peptides (sequences from ID45 to 46 and from ID60 to 61 of this application),
SARS coronavirus protein N peptides (sequences from ID47 to 48 and from ID62 to 63 of this application),
SARS coronavirus protein S peptides (sequences from ID49 to 58 and from ID64 to 73 of this application),
SARS coronavirus protein M peptides (sequences from ID45 to 46 of this application),

Human non-SARS Coronavirus composition. It comprises the following antigen preparations:
Human Coronavirus Group 1 (strain 229E) protein S peptides (sequences from ID173 to 177 of this application),
Human Coronavirus Group 2 (strain OC43) protein S peptides (sequences from ID178 to 182 of this application),

The present method makes use of peptides designed to be pathogen-specific, highly conserved, and independent of HLA haplotypes in the individual under scrutiny. The peptide strategy allows the formulation of effective stimuli specific for any pathogen whose sequence data are present in database. Moreover it allows the formulation, design and use of specific stimuli for any new pathogen as soon as the relevant sequence data are available.

The diagnostic method according to the invention and the method of peptide design can be extended to a wide range of pathologies with a different aethiology:
natural or intentionally produced infections from different sources (such as respiratory or in utero or emerging or post-transplant infections or biological threat agents),
neoplastic diseases, etc. known by at least one antigen.

According to the method, a general protocol of in vitro stimulation of peripheral blood mononuclear cells (PBMC) by Patho-tope arrays is provided. PBMC are at first isolated from venous blood using a gradient separation well known to the skilled man: among the commercially available kits the preferred are: LeucoSep™, by Arnika, Milano and BD Vacutainer™ CPT™ by Becton-Dickinson, CA. Patient and control PBMCs are then stimulated in vitro by the composition of antigens according to the invention, in optimal conditions which represents another embodiment of the invention. After 6-12 hours of incubation, the qualitative and quantitative analysis of T lymphocytes specific for the antigen(s) known to be related, expressed or associated to the pathology to be diagnosed, is performed by detecting the frequency of cytokines producing cells by flow-cytometry or by detecting T-lymphocyte membrane specific antigens or both. The detection of T lymphocyte membrane antigens presence or level of expression is preferably performed on at least one of the antigens selected from the group of: CD3, CD45, CD4, CD8, CD25, CD27, CD38, CD45-RA, CD45-RO, CD69, CCR5, or CCR7 by specific antibodies, preferably monoclonal antibodies all of which are commercially available.

This characterization is performed by adapting a well known method for the selective measurement of antigen specific T lymphocytes and cytokine producing T lymphocytes (Maino V C and Picker I J Cytometry 1998; 34:207-215).

Cytokines measurements is performed by antibodies, preferably by monoclonal antibodies (all of which commercially available) on cytokines which are activated or whose expression is induced or enhanced during the antigen-induced immune response, selected from the group comprising: interferon gamma, IL-2, IL-4, IL-10, TNF-α,. MIP-α, MIP-1β, RANTES, and combinations thereof. Interferon gamma is preferably detected.

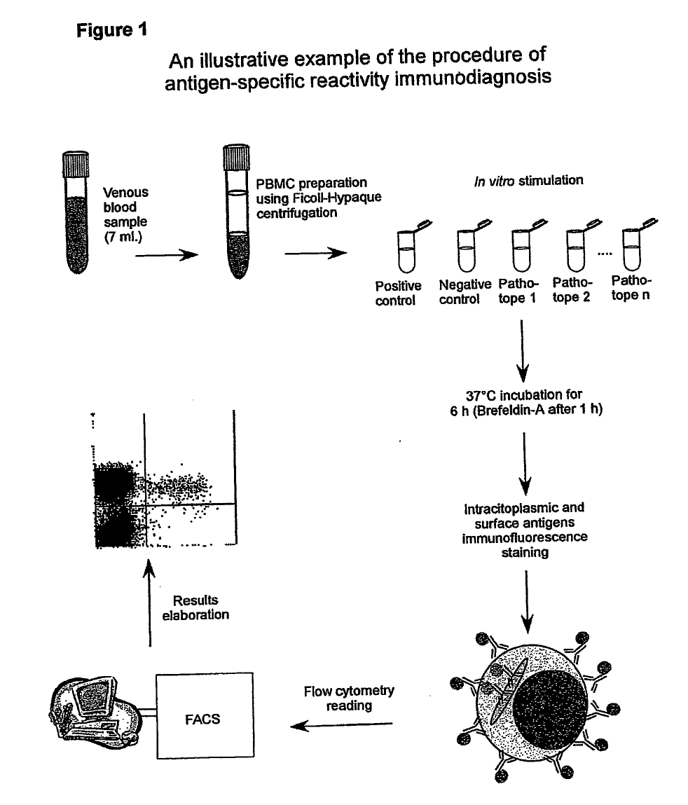

The flow cytometry test described in this invention can be performed using either fresh or cryopreserved PBMCs depending on the Pathotope specificity and on the viability of the frozen samples. Fresh preparations are preferred. The various steps are schematically illustrated in FIG. 1.

For step (i), the PBMC are preferably isolated from a sample of heparinated venous blood (typically 7 ml) through centrifugation on density gradient using a rapid method (as described previously) based on use of tubes with filters for the separation of leukocytes.

Step ii) is performed according to the preferred embodiment outlined in steps 1) through 8) and depicted in the flowchart in FIG. 4.

For step (iii), the method of the invention further comprise the preparation of at least a positive and a negative control, wherein the negative control is represented by unstimulated T-lymphocytes and the positive control is represented by mitogen-stimulated cells. Mix of peptides or antigens to be used in PBMC stimulation may be also provided as ready to use compositions. A negative control is preferably prepared by stimulating cells with a control stimulus such as antigenic extract from non-infected cultures, irrelevant recombinant proteins, or the medium used for peptide dilutions.

As positive controls, PBMCs stimulated with mitogens, such as ionomycin optionally in the presence of PMA (Phorbol Myristate Acetate) is preferably used, since by their use a strong signal of T lymphocyte activation is easy detectable by flow-cytometry. Reagents for negative or positive controls, and Patho-tope arrays preparations can be manufactured as freeze-dryed solutions to be reconstituted at the moment of use.

As described in the examples, the Patho-tope arrays preparations comprise three different categories of antigens (depending on the level of purification), and whose composition is selected according to the kind of analysis needed. They may comprise raw protein extracts, purified or recombinant proteins, or a mix of synthetic peptides.

In steps (iv) and (v) the samples to be examined (PBMC) are placed in contact or incubated with the antigen-specific Patho-tope arrays preparation in vitro, incubated preferably at 37° C. for approximately one hour, and further incubated preferably for about 5 hours in presence of a potent inhibitor of cellular protein secretion, such as monensin or Brefeldin-A.

In step (vi) the selective immunofluorescent staining is performed by using monoclonal antibodies on control and Patho-tope arrays preparation-stimulated T-lymphocyte cultures by standard techniques. For the selective measurement of the antigen specific (Ag-Sp) T lymphocytes, the following antibodies are used: A) monoclonal antibodies for T lymphocytes specific cell surface markers and subpopulations thereof; B) monoclonal antibodies for cytokines accumulated intracellularly in the T-lymphocytes after stimulation with the antigen; C) mixtures of A)and B).

In order to discriminate T lymphocyte populations according to the method of the invention PBMC are stained with a mixture of labelled monoclonal antibodies recognizing at least one marker present on the surface T of the lymphocytes (e.g. typically CD3, CD45 and mixtures thereof). When labelled primary antibodies are not available secondary labelled antibody can be used as known by the skilled man.

Antibodies used to measure and characterize a single population or different stages of differentiation and/or activation of T lymphocytes are typically anti-CD3, anti-CD45, and related mixtures as a minimal configuration, to which at least one antigen measurement selected from the group comprising: CD4, CD8, CD25, CD27, CD38, CD45-RA, CD45-RO, CD69, CCR5, and CCR7 antigen may be added. As a preferred embodiment, with the aim to quantitatively detect the response to the stimulation, specific antibodies for intracellular IFN-gamma are used to estimate the production of this cytokine as a sign of antigen-driven response. As a signal of quantitation of the T-lymphocyte activation, other intracellular cytokines, comprising IL-2, IL4, IL-10, TNF-α, MIP-1α, MIP-1β, RANTES, are also used.

In step (vii) the sample fluorescence is acquired and analysed with a flow-cytometer using standard laboratory procedures. As a specific embodiment, the invention relates to the flow cytometric analysis of T lymphocyte differentiation markers and of intracytoplasmatic cytokines that allows the acquisition of both quantitative and qualitative results.

Finally, in step (viii) the response to the test is expressed as a quantitative (presence/absence of the Ag-Sp T lymphocytes) or quantitative response (frequency of responder cells for unit of volume of blood) as described in detail in the examples. The flow cytometry sensitivity limits allow to find differences in percentage below 0,02%. For GLP practice, the interval of normality has to be set within each laboratory.

In summary the method disclosed in the present invention
provides both qualitative and quantitative results, expressed either by frequency or by absolute values of antigen-specific T lymphocytes present in the peripheral blood;
allows the characterization of the T cell subset or the effector stage known to respond to a specific Patho-tope (eg. CD4 or CD8 T cells, CD45-RA and CD27, etc.), resulting in a more specific and sensitive identification of the T cell response for diagnostic purpose and minimizing the aspecific background of the diagnostic test;
provides specific arrays comprising a panel of pathology-specific epitopes (Patho-topes) covering many different set of pathologies. Such arrays have been designed, tested and validated. Such arrays are assembled and manufactured as a ready-to-use pathology-specific arrays which can be assembled in ready to use diagnostic kits;
it can be performed in less than 24 hours, sometimes in less than 8 hours, and is effective also using cryopreserved samples.

According to further embodiments of the invention kits are provided comprising the compositions of the specific mix of peptides or of purified antigens or pathogen lysates (Patho-tope arrays) preparations according to the invention and further comprising:
negative and positive controls;
a panel of pathogen-specific stimuli, optionally a panel of vaccine-specific stimuli, in the presence of a costimulus such as anti CD28 and CD49d monoclonal antibodies;
washing and permeabilization reagents;
reagents as mixtures of monoclonal antibodies to detect T-lymphocyte surface markers or cytokine production;
optionally pipets, tubes and others laboratory items;
detailed instruction for the set-up and the interpretation of the test.

In a specific embodiment of the invention, the immuno-diagnostic test described here is used to detect the appearance or the re-emerging of all infectious, autoimmune or neoplastic diseases that generate a specific T cell response. Since the induction of an effective response by T lymphocytes needs only a few days, and precedes the appearance of detectable antibodies by some weeks, the method described in this invention has the following advantages:
the frequency of the Ag-Sp T lymphocytes is correlated to the antigenic exposition, and it can be therefore used, for example, to detect the sub-clinical exposition to an infectious agent;
elevated levels of Ag-Sp T lymphocytes are present during the acute phase of disease, and their absence or reappearance can be a index of resolution or relapse of the pathology;
Ag-Sp T lymphocytes monitoring may be used in order to evaluate the effectiveness of a chemotherapy or vaccination protocol.

In the following examples, the procedure of the Ag-Sp T lymphocyte immuno-diagnosis test, the elaboration in a diagnostic response of the results, and the single issues relevant to the antigenic preparations are reported in detail as mere examples describing the present invention, and not limiting it in any way to the particular issue.

A particularly valuable embodiment of the invention, is represented by the definition of several pathology-specific Patho-tope arrays preparations are described, allowing a differential diagnosis approach in different pathology situations including:
different natural or intentionally produced infections (such as respiratory or in utero or emerging or post-transplant infections or biological threat agents),
neoplastic diseases.

Preferred Pathotope arrays compositions are peptide compositions, represented by a set of synthetic peptides, specific for a particular antigen, which have been designed and validated as particularly efficient in specific T-lymphocytes stimulation, either CD4⁺ or CD8⁺. Peptide sequences are enlisted as SEQ ID NO 1-182 in the Sequence Listing.

To the purpose of the present invention the term composition is referred to a mix of at least one or preferably at least two or preferably at least three or preferably more than three different peptides derived from the same or different protein or antigen or to a mixed composition comprising any other kind of antigen as defined above (raw antigenic extract, isolated proteins, etc.).

Peptides according to the invention are defined as sequences comprising at least 9 consecutive aminoacids derived from each of the sequences enlisted as SEQ ID NO 1 to SEQ ID NO 182. However the term peptide encompasses also peptides comprising additional aminoacids at the N- or C-terminus, for a maximum length of 30 aminoacids, or more preferably 29, or 28, or 27, or 26, or 25, or 24, or 23 or 22 or 21 or 20 or 19, or 18, or 17, or 16, or 15, or 14, or 13, or 12, or 11, or 10 aminoacids, besides the 9 consecutive aminoacids defined above. Additional aminoacids are derived from relevant sequences in GenBank. Resulting peptides are selected so as to maintain the same properties of specificity and promiscuity of the peptides identified in the sequence listing. GenBank accession numbers corresponding to the relevant antigen from which each peptide has been derived in the sequence listing, are reported in the following table:

| | | GenBank | | |
|---|---|---|---|---|
| SEQ ID N^{o} | Code | Accession | n^{o} | Prot [symbol] |
| SEQ ID NO 1-20 | HIV-GAG | AAP35014 | | gag p [symbol] |
| SEQ ID NO 21-43 | CMV-p66 | P29839 | | Hum [symbol] |
| SEQ ID NO 44, 59 | SCV-E | AAS44718 | | small |
| SEQ ID NO 45-46, 60-61 | SCV-M | AAP97886 | | M pr [symbol] |
| SEQ ID NO 47-48, 62-63 | SCV-N | AAS48576 | | nucle |
| SEQ ID NO 49-58, 64-73 | SCV-S | AAS10463 | | spike |
| SEQ ID NO 74-83 | Ba-PA | 2005240 | | Bacill |
| SEQ ID NO 84-85 | OPV-A10L | NP_042158 | A10L | [Vari [symbol] |
| SEQ ID NO 86-87 | OPV-A27 | NP_042175 | A27L | [Vari [symbol] |
| SEQ ID NO 88-90 | OPV-A36R | NP_042184 | A36R | [Vari [symbol] |
| SEQ ID NO 91-92 | OPV-C7L | NP_042071 | C7L | [Vari [symbol] |
| SEQ ID NO 93 | OPV-F8L | NP_042142 | F8L | [Vari [symbol] |
| SEQ ID NO 94-95 | OPV-E3L | AAB29618 | E3L | [Vari [symbol] |
| SEQ ID NO 96-97 | OPV-H2L | NP_042108 | H2L | [Vari [symbol] |
| SEQ ID NO 98-99 | OPV-H6R | NP_042113 | H6R | [Vari [symbol] |
| SEQ ID NO 100-101 | OPV-K1L | NP_042099 | K1L | [Vari [symbol] |
| SEQ ID NO 102-103 | OPV-M1R | NP_042117 | M1R | [Vari [symbol] |
| SEQ ID NO 104-122 | AFP | NP_001125 | | alpha |
| SEQ ID NO 123-142 | PSA | AAA60193 | | prost [symbol] |
| SEQ ID NO 143-157 | MAGE-3 | NP_005353 | | mela [symbol] |
| SEQ ID NO 158-172 | NY-ESO-1 | NP_001318 | | New |
| SEQ ID NO 173-177 | HuCoV-1 | NP_073551 | | spike |
| SEQ ID NO 178-182 | HuCoV-2 | NP_937950 | | spike |

For the detection of infectious diseases the following agent specific compositions may be used alone or in combination with other peptide compositions or with other mixed compositions:

HIV Compositions.

The HIV gag composition comprises as the immunoreactive principle, at least one or preferably three peptides comprising at least 9 consecutive aminoacids derived from any of the sequences from SEQ ID NO 1 to SEQ ID NO 20. This composition may be combined with antigenic preparation for the detection of other HIV proteins, in particular HIV tat and HIV nef.

CMV Composition

The CMV composition comprises as the immunoreactive principle, at least one or preferably three peptides comprising at least 9 consecutive aminoacids derived from any of the sequences from SEQ ID NO 21 to SEQ ID NO 43.

SARS Compositions

The SARS coronavirus infection composition comprises as the immunoreactive principle, at least one or preferably three peptides comprising at least 9 consecutive aminoacids derived from any of the sequences from SEQ ID NO 44 to SEQ ID NO 73.

In particular the detection of E-protein specific T-lymphocytes is performed with a composition comprising at least one of the SARS coronavirus E-protein derived peptides selected from: SEQ ID NO 44 and SEQ ID NO 59; the detection of M-protein specific T-lymphocytes is performed with the composition comprising at least one M-protein derived peptide selected from: SEQ ID NO 45, SEQ ID NO 46, SEQ ID NO ID60, SEQ ID NO 61; the detection of N-protein specific T-lymphocytes is performed with a composition comprising at least one N-protein derived peptide selected from: SEQ ID NO 47, SEQ ID NO 48, SEQ ID NO 62, SEQ ID NO 63; the detection of S-protein specific T-lymphocytes is performed with a composition comprising at least one S-protein derived peptide selected from the group consisting of: SEQ ID NO 49 to SEQ ID NO 58 and the group consisting of SEQ ID NO 64 to SEQ ID NO 73.

For diagnosis of SARS coronavirus infection a composition specific for the Asian population (A-SARS) has been designed which comprises at least three peptides from SEQ ID NO 44 to SEQ ID NO 58; while for the Caucasian population (B-SARS) the composition comprises at least one or preferably two, even more preferably three peptides selected from the group of: SEQ ID NO 59 to SEQ ID NO 73. To discriminate the SARS infection from the common Coronavirus-induced cold, peptides built on proteins from two different non-SARS inducing strains (229E, Group1: SEQ ID NO 173 to SEQ ID NO 177; OC43, Group 2: SEQ ID NO 178 to SEQ ID NO 182) were also included.

B.anthracis Composition

A composition specific for immunodiagnosis of B.anthracis infection comprises at least one, preferably two, even more preferably three peptides selected from the group consisting of peptides comprising at least 9 consecutive aminoacids comprised within the following sequences: SEQ ID NO 74 to SEQ ID NO 83. Preferably the peptides are selected among SEQ ID NO 74 to SEQ ID NO 83.

Orthopox Composition

A composition specific for immunodiagnosis of orthopoxviridae infection or vaccination comprises as the immunoreactive principle, at least one, preferably two, even more preferably three peptides comprising at least 9 consecutive aminoacids comprised within the following sequences: from SEQ ID NO 84 to SEQ ID NO 103.

For detection of T-lymphocytes specific for tumor antigens and for the diagnosis of neoplasy the following compositions have been designed:
an alfafetoprotein specific composition comprises as the immunoreactive principle, at least one, preferably two, even more preferably three peptides comprising at least 9 consecutive aminoacids comprised within the sequences from SEQ ID NO 104 to SEQ ID NO 122. Preferably the peptides are those with sequence from SEQ ID NO 104 to SEQ ID NO 122;
a PSA (Prostatic Specific Antigen) specific composition comprises as the immunoreactive principle, at least one, preferably two, even more preferably three peptides comprising at least 9 consecutive aminoacids comprised within sequences from SEQ ID NO 123 to SEQ ID NO 142.
a MAGE (melanoma-associated antigen 3) specific composition comprises as the immunoreactive principle, at least one, preferably two, even more preferably three peptides comprising at least 9 consecutive aminoacids comprised within sequences from: SEQ ID NO 143 to SEQ ID NO 157; the peptides are preferably chosen among sequences from SEQ ID NO 143 to SEQ ID NO 157.
NY-eso (New York esophageal squamous cell carcinoma 1) specific composition comprises as the immunoreactive principle, at least one, preferably two, even more preferably three peptides comprising at least 9 consecutive aminoacids comprised within sequences from: SEQ ID NO 158 to SEQ ID NO 172; the peptides are preferably chosen among sequences from SEQ ID NO 158 to SEQ ID NO 172.

In oncologic diagnosis the method disclosed is particularly useful in the detection and diagnosis of melanoma, hepatocarcinomas, prostatic tumors, hesophageal tumors or in any other tumor wherein at least one of the above cited markers is overexpressed.

Combination of at least one of the composition of the invention with different antigen preparations (either as purified proteins or lysate) are also used in the method of the invention. Preferred combinations designed to allow a better specificity and a higher responsiveness among individuals are the following, grouped according to the specific pathology or group of pathologies to be diagnosed.

Biological threat agents Patho-tope Array. It refers to differential diagnosis of the following agents, with the indicated antigens, peptides or lysates preparation:

Ortho-Poxviruses,

Protein A10L peptides (from sequence ID84 to 85 of this application)
Protein A27 peptides (from sequence ID86 to 87 of application)
Protein A33R peptides (from sequence ID88 to 90 of this application)
Protein C7L peptides (from sequence ID91 to 92 of this application)
Protein D8L peptides (from sequence ID93 of this application)
Protein E3L peptides (from sequence ID94 to 95 of this application)
Protein H3L peptides (from sequence ID96 to 97 of this application)
Protein H6R peptides (from sequence ID98 to 99 of this application)
Protein K1L peptides (from sequence ID100 to 101 of this application)
Protein M1R peptides (from sequence ID102 to 103 of this application)

Anthrax (B.anthracis)
Protective antigen Protein peptides (from sequence ID74 to 83 of this application);

Plague (Yersinia pestis)
Capsular F1 antigen (QED Bioscience, San Diego, Calif.)

Tularemia (Francisella tularensis)
LPS antigen (QED Bioscience, San Diego, Calif.).

SARS coronavirus composition. It comprises the following antigen preparations:
SARS coronavirus protein E peptides (sequence ID44 and 59 of this application)
SARS coronavirus protein M peptides (sequences from ID45 to 46 and from ID60 to 61 of this application),
SARS coronavirus protein N peptides (sequences from ID47 to 48 and from ID62 to 63 of this application),
SARS coronavirus protein S peptides (sequences from ID49 to 58 and from ID64 to 73 of this application),
SARS coronavirus protein M peptides (sequences from ID45 to 46 of this application),
SARS coronavirus recombinant protein E (Biodesign Int., Saco, Me.),
SARS coronavirus recombinant protein M (Biodesign Int., Saco, Me.),
SARS coronavirus recombinant protein Nucleocapsid aa.1-49 (Biodesign Int.,Saco, Me.),
SARS coronavirus recombinant protein Nucleocapsid aa.192-220(Biodesign, Me.).

Human non-SARS Coronavirus composition. It comprises the following antigen preparations:
Human Coronavirus Group 1 (strain 229E) protein S peptides (sequences from ID173 to 177 of this application),
Human Coronavirus Group 2 (strain OC43) protein S peptides (sequences from ID178 to 182 of this application),

Respiratory infections Patho-tope Array. It refers to the differential diagnosis of the following agents: Influenza A virus, Influenza B virus, Parainfluenza virus, Respiratory Syncytial Virus, SARS coronavirus, Echovirus II, Coxsackie virus, Adenovirus) Legionella pneumophila, Mycoplasma pneumoniae, Chiamidia pneumoniae, with the following antigens, peptides or lysates preparation:

Influenza A virus composition. It comprises the following antigen preparations:
Influenza A virus (H3N2) antigen lysate (Biodesign Int., Saco, Me.; Research Diagnostic Inc., Flanders, N.J.)
Influenza Avirus (H1N1) antigen lysate (Biodesign Int., Saco, Me.; ABI, Columbia MD; Research Diagnostic Inc., Flanders, N.J.)

Influenza B virus composition. It comprises the following antigen preparations:
a Influenza B virus (Hong Kong) antigen lysate (Biodesign Int., Saco, Me.)
Influenza B virus (Victoria) antigen lysate (Biodesign Int., Saco, Me.; Research Diagnostic Inc., Flanders, N.J.)
Influenza B virus (Tokio) antigen lysate (Biodesign Int., Saco, Me.; Research Diagnostic Inc., Flanders, N.J.)
Influenza B virus (Qiengdao) antigen lysate (Biodesign Int., Saco, Me.)
influenza B virus (Lee) antigen lysate (ABI, Columbia Md.)

Parainfluenza virus composition. It comprises the following antigen preparations:
Parainfluenza virus (group 1) antigen lysate (Biodesign Int., Saco, Me.; Research Diagnostic Inc., Flanders, N.J.)
Parainfluenza virus (group 2) antigen lysate (Biodesign Int., Saco, Me.; Research Diagnostic Inc., Flanders, N.J.)
Parainfluenza virus (group 3) antigen lysate (Biodesign Int., Saco, Me.; Research Diagnostic Inc., Flanders, N.J.)
Parainfluenza virus (group 4) antigen lysate (Biodesign Int., Saco, Me.) Respiratory Syncytial Virus composition. It comprises the following antigen preparations:
Respiratory Syncytial Virus (RSV, ceppo A2) antigen lysate (Biodesign Int., Saco, Me.; ABI, Columbia Md.; Research Diagnostic Inc., Flanders, N.J.)

SARS coronavirus composition. It comprises the following antigen preparations:
SARS coronavirus protein E peptides (sequence ID44 and 59 of this application)
SARS coronavirus protein M peptides (sequences from ID45 to 46 and from ID60 to 61 of this application),
SARS coronavirus protein N peptides (sequences from ID47 to 48 and from ID62 to 63 of this application),
SARS coronavirus protein S peptides (sequences from ID49 to 58 and from ID64 to 73 of this application),
SARS coronavirus protein M peptides (sequences from ID45 to 46 of this application),
SARS coronavirus recombinant protein E (Biodesign Int., Saco, Me.),
SARS coronavirus recombinant protein M (Biodesign Int., Saco, Me.),
SARS coronavirus recombinant protein Nucleocapsid aa.1-49 (Biodesign Int.,Saco, Me.),
SARS coronavirus recombinant protein Nucleocapsid aa.192-220(Biodesign, ME).

Human non-SARS Coronavirus composition. It comprises the following antigen preparations:
Human Coronavirus Group 1 (strain 229E) protein S peptides (sequences from ID173 to 177 of this application),
Human Coronavirus Group 2 (strain OC43) protein S peptides (sequences from ID178 to 182 of this application),

echovirus 11 composition. It comprises the following antigen preparations:
echovirus 11 antigen lysate (Biodesign Int., Saco, Me.)

Coxsackie virus composition. It comprises the following antigen preparations:
Coxsackie B6 antigen lysate (Biodesign Int., Saco, Me.),
Coxsackie A9 antigen lysate (Biodesign Int., Saco, Me.)
Coxsackie A16 antigen lysate (Biodesign Int., Saco, Me.)

Adenovirus composition. It comprises the following antigen preparations:
adenovirus (tipo 3) antigen lysate (Biodesign Int., Saco, Me.);
adenovirus (tipo 6) antigen lysate (Biodesign Int., Saco, Me., Research Diagnostic Inc., Flanders, N.J.),
adenovirus (tipo 21) antigen lysate (Biodesign Int., Saco, Me.);

Legionella pneumophila antigen (Trinity Biotech Plc, Wicklow, Ireland);

Mycoplasma Pneumoniae antigen lysate (Diesse, Florence, Italy);

Chiamidia Pneumoniae antigen lysate (Mast Diagnostica, Reinfeld, Germany).

Enteric infections Patho-tope Array. It refers to differential diagnosis to the following agents, with the indicated antigens, peptides or lysates preparation (between parenthesis):
Shigella groups A, A1, B, C, C1, C2 antigens (BD Diagnostic Systems, Sparks, Md.);
Salmonella groups A, 0 antigens (BD Diagnostic Systems, Sparks, Md.);
Enterovirus 70 antigen lysate (Biodesign Int., Saco, Me.);
HAV antigen lysate (Research Diagnostic Inc., Flanders, N.J.);
HEV Hepatitis E Virus ORF2 antigen (Research Diagnostic Inc., Flanders, N.J.);
Helicobacter pylori HPSa antigen (Meridian Bioscience, Cincinnati, Ohio);
Clostridium difficile Toxin A antigen (Meridian Bioscience, Cincinnati, Ohio).

Sexually transmitted diseases Patho-tope Array. It refers to differential diagnosis to the following agents, with the indicated antigens or peptides or lysates preparation (between parenthesis):
Treponema pallidum, p15 recomb. antigen (Research Diagnostic Inc., Flanders, N.J.);
Treponema pallidum, p17 recomb. antigen (Research Diagnostic Inc., Flanders, J);
Treponema pallidum, p45 recomb. antigen (Research Diagnostic Inc., Flanders, N.J.);
Treponema pallidum, TmpA recomb. antigen (Res. Diagnostic Inc., Flanders, N.J.);
HPV L1, capsid antigen recombinant protein (Res. Diagnostic Inc., Flanders, N.J.)
Candida albicans, mixed antigen (IBL Inc., Minneapolis Minn.);
HSV2, antigen lysate (Tebu-Bio, Le Perray en Yvelines, France; Res. Diagnostic Inc., Flanders, N.J.);
HBV, HBeAg recombinant antigen (Research Diagnostic Inc., Flanders, N.J.);
HBV, Core recombinant antigen (Research Diagnostic Inc., Flanders, N.J.);
HBV, HBsAg recombinant antigen (Research Diagnostic Inc., Flanders, N.J.);
HIV-1, protein Gag peptides (from sequence ID 1 to 20 of this application);
HIV-1, antigen lysate (Tebu-Bio, Le Perray en Yvelines, France);
HIV-2, antigen lysate (Tebu-Bio, Le Perray en Yvelines, France);
HIV-1, recombinant protein Gag (Research Diagnostic Inc., Flanders, N.J.);
HIV-1, recombinant protein Nef (Research Diagnostic Inc., Flanders, N.J.);
HIV-1, recombinant protein Env (Research Diagnostic Inc., Flanders, N.J.).

In utero infections Patho-tope Array. It refers to differential diagnosis to the following agents, with the indicated antigens, peptides or lysates preparation:
Toxoplasma gondii, lysate (Research Diagnostic Inc., Flanders, N.J.)
Toxoplasma gondii, Tachyzoites antigen (Research Diagnostic Inc., Flanders, N.J.)
Rubella, recombinant protein (Research Diagnostic Inc., Flanders, N.J.)
CMV (AD169), antigen lysate (Biodesign Int., Saco, Me.; (ABI, Columbia Md.)
CMV (AD169), pp65 recomb. protein (Austral Biologicals, San Ramon, Calif.;

Biodesign Int., Saco, Me.; Research Diagnostic Inc., Flanders, N.J.)
CMV (AD169), pp150 recomb. protein (Biodesign Int., Saco, Me.)
CMV (AD169), pp28 recomb.protein (Biodesign Int., Saco, Me.)
CMV (AD169), pp38 recomb. protein (Biodesign Int., Saco, Me.)
CMV (AD169), p50 recomb. protein (Austral Biologicals, San Ramon, Calif.;

Research Diagnostic Inc., Flanders, N.J.)
CMV (C194), gB recomb. protein (Biodesign Int., Saco, Me.)
Peptides as in sequences ID from 21 to 43 of this application
HSV-1 gD recombinant protein (Research Diagnostic Inc., Flanders, N.J.)
HSV-1 gG recombinant protein (Research Diagnostic Inc., Flanders, N.J.)
HSV-1 viral lysate (Tebu-Bio, Le Perray en Yvelines, France; Research Diagnostic Inc., Flanders, N.J.)
VZV antigen lysate (Research Diagnostic Inc., Flanders, N.J.).

Post-transplant infections Patho-tope Array. It refers to differential diagnosis to the following agents, with the indicated antigens, peptides or lysate preparation:
CMV as described for the in utero infections;
EBV (B95-8) antigen lysate (Tebu-Bio, Le Perray en Yvelines, France);
HSV-1 as described for the in utero infections.

Blood-bome infections Patho-tope Array. It refers to differential diagnosis to the following agents, with the indicated antigens, peptides or lysates preparation:
HIV-1 as described for the Sexually transmitted diseases,
HCV Core recombinant protein (Research Diagnostic Inc., Flanders, N.J.)
HCV p22 nucleocapsid recombinant protein (Res. Diagnostic Inc., Flanders, N.J.)
HCV NS3 recombinant protein (Research Diagnostic Inc., Flanders, N.J.)
HCV NS4 recombinant protein (Research Diagnostic Inc., Flanders, N.J.)
HBV as described for the in utero infection
HDV delta antigen, recombinant (Cortez Diagnostics, Calabasas, Calif.)
HGV antigen, recombinant (Cortez Diagnostics, Calabasas, Calif.)
HHV-8 antigen lysate (Tebu-Bio, Le Perray en Yvelines, France).

Neoplastic diseases Pathotope array
AFP peptides (from sequence ID104 to 122 of this application)
PSA peptides (from sequence ID123 to 142 of this application)
MAGE-3 peptides (from sequence ID143 to 157 of this application)
NY-ESO-1 peptides (from sequence ID158 to 172 of this application)

### EXPERIMENTAL PART

### EXAMPLE 1

### Procedure of Execution of the Patho-tope Arrays T Lymphocytes Immuno-diagnosis Test by Flow-cytometry.

The following specific monoclonal antibodies for human antigens were used for the execution of the test: purified anti-CD28e anti-CD49d as co-stimulator factors during the cellular cultures; anti-IFN-gamma conjugated with fluorescein (FITC);

anti-CD3 conjugated with phycoerythrin (PE); anti-CD45 conjugated with phycoerythrin-cyanin-5(Cy-5) and a isotypic control (IgG1) conjugated with FITC.

The antibodies are used at the concentration of 0,25 μg/ml. Each new batch of antibodies was tested, and the antibodies mixtures (mix) were set-up ready for use in 1 mL microcentrifuge tubes. Specifically, each antibody was used in saturating conditions to exclude differences in the samples during the staining. The tubes were then placed in a Speedvac freeze-dryer until complete evaporation of the solvent (20 min). At the moment of the use each mix was reconstituted by adding saline, and was aliquoted to the tubes containing the cells to be analysed. Peripheral blood mononuclear cells (PBMC) were isolated from 7 ml of venous blood by centrifugation on density gradient (Ficoll-Hypaque, Pharmacia, Uppsala, Sweden) using a rapid method based on leucocytes separation in 14 ml tubes with a filter (LeucoSepTM, ARNIKA, Milan). After 2 washes in PBS, the pellet was resuspended in 3 ml of complete medium (RPMI 1640 with HEPES 25 mM, 10% v/v FCS, 2 mM L-Glutamine, 10 U/ml penicillin/streptomycin) at a concentration of 0.5-2×10⁶ cells/ml. Moreover, 500 μl of the cellular suspension was then dispensed in microcentrifuge tubes. Two control tubes (not stimulated and with mitogen stimulus), and one or more tubes containing the Ag-Sp antigen-specific preparations were used, depending on the particular analysis to be performed. The spontaneous production of cytokines was checked in every test by incubating the cells with the co-stimululatory antibodies (anti CD28 and CD49d) (non-stimulated control). PMA (50 ng/ml)+ionomycin (10 μ/ml) were used as a positive control. Negative and positive controls may be manufactured as freeze-dried preparations to be reconstituted just before use. The samples were then incubated at 37° C. for approximately one hour followed by a further 5 hours in in presence of 10 μg/ml of Brefeldin-A (Sigma, St. Louis, Mo.), a potent inhibitor of the cellular secretion.

In order to complete the FACS staining, control or stimulated cells were washed twice in PBS (Dulbecco's phosphate-buffered saline) medium containing 1% of bovine serum albumin (BSA) and 0,1% of sodium azide. The cells were then stained for 15 min at 4° C. with the mix of specific monoclonal antibodies for membrane antigens (CD3 and CD45) as previously described. The samples were then fixed in 1% paraformaldehyde for 10 minutes at room temperature, and incubated with IFN-gamma-specific antibodies in PBS containing BSA 1% and saponin 0,5%. The cells were washed twice in PBS containing BSA 1% and saponin 0,1% and finally resuspended in PBS to be acquired by flow-cytometry (e.g. FACScalibur, Becton Dickinson, Calif.). Non-specific staining was determined by isotypic control monoclonal antibodies, in order to subtract any background. The samples were then analysed by flow-cytometry using standard methods. For each sample, 1×10⁵ lymphocyte (CD45⁺) events were acquired, in order to assure the adaquete representation of all cell populations to allow for the significance statistic analyses necessary for the elaboration of a diagnostic response.

### EXAMPLE 2

### Elaboration of Results and Formulation of a Diagnostic Response

A T-cell response profile was developed for several individuals (see FIG. 1a). A marked, specific response to CMV antigens was seen in each of the healthy donor panels. There was a large individual variability, but sample duplicates confirmed specificity. Neither of these results were unexpected, as the prevalence of seropositivity for CMV in Italy is quite high and the response levels were expected to vary, depending on the individual. Pathogen-infected or recently vaccinated individuals were used as controls to confirm the reactivity of the antigen mixes for the response panel. As shown in FIG. 1b, a robust response was observed in infected or vaccinated individuals for their respective pathogens.

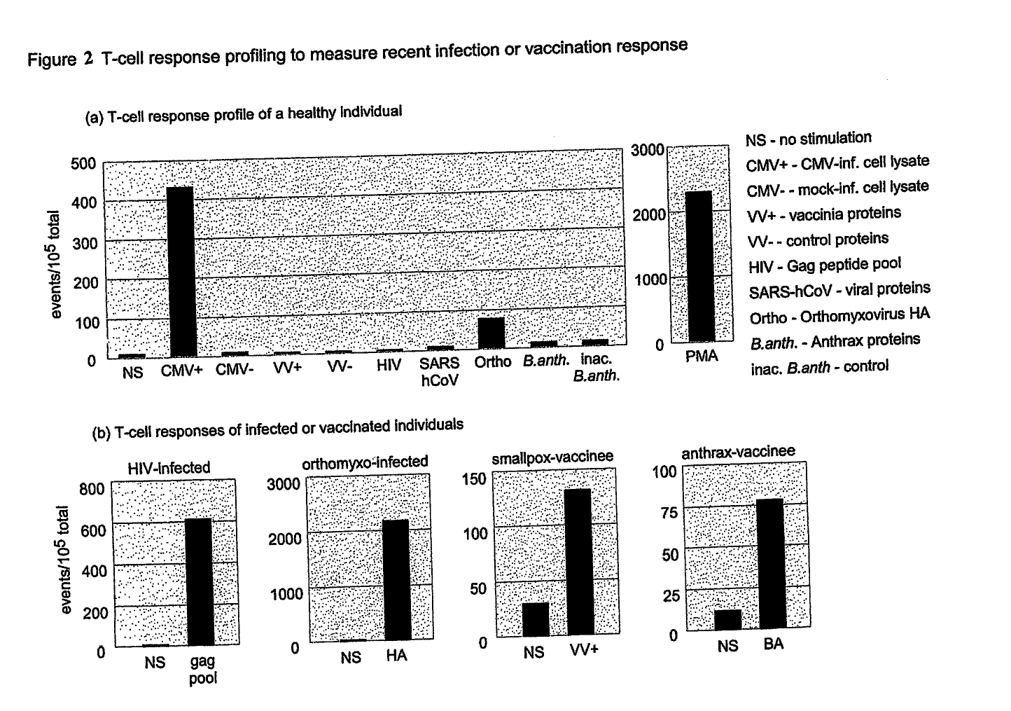

A small, but reproducible response was seen to the recombinant SARS protein pool in a number of healthy donors (FIG. 2a). Selected epitopes in our preparation are not unique to the class IV coronavirus (SARS-hCoV), but are instead conserved among the other classes of coronaviruses which can cause the common cold. It appears that the recombinant proteins for SARS-COV E and N2 contain cross-reactive epitopes as these proteins stimulated a response which was above assay background.

### EXAMPLE 3

### Description of the Procedure using as Ag-Sp Formulations a Raw Antigenic Extract, Recombinant Proteins, or Mixtures of Peptides

In this experiment different antigenic preparations were used: (a) raw protein extract, (b) purified or recombinant proteins, (c) mixtures of peptides.
(a) As an example, the methodology of purification of antigenic extract from fibroblast or VERO cells infected with the vaccinia virus is reported. The cells susceptible to the infection were transferred in a tube containing vaccinia virus to a multiplicity of infection (MOI)=100. The incubation was performed at 37° C. until 50% of the cytopathic effect was found. At that time, a centrifugation to 850 g×15 min was performed followed by fixation in PFA 2% for 10 min. at 4° C. After three washes in PBS, the cell pellets were sonicated for 20 min at 4° C. in PBS, centrifuged at 850 ×g for 15 min at 4° C. and aliquoted at −20° C. Each new antigenic preparation batch must be checked to find the best working dilution to be used in the immuno-diagnosis Ag-Sp T lymphocyte test. As a negative control, raw antigenic extracts of non-infected cells were produced following the same procedure. Similar results may be obtained using commercial antigenic preparations used for ELISA tests. For example, an antigenic viral preparation produced by Maine Biotechnological Service, Portland, Me., was used as a vaccinia virus-specific stimulus (results are shown in FIG. 1b). For example, an antigenic viral preparation produced by Biowhittaker, Walkersville, Md., was used as a CMV-specific stimulus (results are shown in FIG. 1a).
(b) As an example, the use of promiscuous peptides selected on the conserved region of HIV-Gag protein is described. These epitopes were defined considering both HIV intra-clades variability for the selected epitopes, and the processing rules of proteins mediated by the proteasome. All synthetic peptides were purified by inverted phase chromatography up to >90%.The sequences and the purity of peptides was confirmed by mass spectrometry. These peptides were used as antigenic stimuli as mixtures of peptides containing also co-stimulating antibodies. The results obtained with the Ag-Sp preparation are reported in FIG. 1. In particular, selected peptides for this experiment comprises SEQ IDNO 1-20 from the HIV-1 gag protein as described in the sequence listing:

The Ag-Sp preparations have been diluted or used at concentration in the range of 1 μg/ml. Moreover, Ag-Sp preparations contained, as costimuli, anti CD28 and anti-CD49d antibodies at a final concentration of 1 μg/ml. Every new stock of Ag-Sp+tubes has been tested and aliquoted in microcentrifuge tubes. The tubes have been placed in a freeze-dryer (Speedvac) until complete evaporation of the liquid (20 min) is obtained. Each mix must be reconstituted before use by adding DMSO (final concentration 0.1%) in isotonic salt solution. The cells to be analysed are then added to the tube.

### EXAMPLE 4

### Selection of a Mixture of Peptides as Patho-tope Arrays Formulation to be used for the Immuno-diagnosis of SARS and of Other Infectious Pathologies

The definition of mixtures of peptides (Patho-tope arrays) used to perform an immuno-diagnostic test by Ag-Sp T lymphocytes is reported for the following infectious pathologies: i) CMV, ii) SARS, iii) smallpox, iv) B.anthracis for which at least one associated antigen was known.

In this example the mixtures of peptides (Patho-tope arrays) used to perform an immuno-diagnostic test by Ag-Sp T lymphocytes has been defined for cytomegalovirus (CMV). Peptides have been designed starting from the consensus sequence of protein p66. The Patho-tope array, specific for CD8 T lymphocytes, comprises the 15 mers mixture corresponding to seq IDNO 21 to SEQ ID NO 43 in the sequence listing.

The mixtures of peptides (Patho-tope arrays) used to perform the immuno-diagnostic test of the invention has been also applied to detection of SARS human coronaviruses (SCoV). Peptides have been designed from the sequences of proteins S, M, E and N. With reference to the geographic distribution of the epidemic, a selection based on the genetic characteristics of the Asian and Caucasian population has been performed. Two Patho-tope arrays, specific for CD8 T lymphocytes, have been designed. In particular, a composition A comprising the 15 mer sequences from SEQ ID NO 44 to SEQ ID NO 58 specific for the detection of the SARS coronavirus in the Asian population and a composition B comprising the 15 mer sequences from SEQ ID NO 59 to SEQ ID NO 73, specific for the detection of the SARS coronavirus in the Caucasian population.

The mixture of peptides (Patho-tope Array) used to perform an immuno-diagnostic test by Ag-Sp T lymphocytes for Bacillus anthracis (Ba) Patho-topes Array, is specific for CD4 T lymphocytes and comprised the 15 mers from SEQ ID NO 74 to SEQ ID NO 83.

The mixtures of peptides (Patho-tope Array) used to perform an immuno-diagnostic test by Ag-Sp T lymphocytes for orthopoxvirus (OPV) including smallpox Patho-tope Array is specific for CD8 T lymphocytes, and comprised the 15 mers from SEQ ID NO 84 to SEQ ID NO 103.

### EXAMPLE 5

### Selection of a Mixture of Peptides in a Patho-topes Array Formulation for the Immuno-diagnosis of Neoplastic Pathologies with Known Tumor-associated Antigens

The mixture of peptides (Patho-topes Array) used to perform the immuno-diagnostic test of the invention on antigen-specific T lymphocytes has been designed also for the detection of neoplastic pathologies and comprised peptides derived from the following tumor-associated antigens: i) alpha-fetoprotein, ii) PSA, iii) MAGE-3, iv) NY-ESO-1. As described in example 3, this procedure and the related application can be extended to any neoplastic pathology for which one associated antigens is known.

The peptides have been designed from the aminoacid sequence of alpha-fetoprotein (AFP) available in GenBank (NP_{—}001125). The following Patho-topes Array, specific for CD8 T lymphocytes, comprises 9 mers from SEQ ID NO 104 to SEQ ID NO 122 of the sequence listing. The mixtures of peptides (Patho-topes Array) to detect PSA Ag-Sp T lymphocytes, was specific for CD8 T lymphocytes and comprised 9 mers from SEQ ID NO 123 to SEQ ID NO 142.

The mixture of peptides (Patho-topes Array) to detect MAGE-3 Ag-Sp T lymphocytes, was specific for CD8 T lymphocytes and comprised 9 mers from SEQ ID NO 143 to SEQ ID NO 157.

The mixtures of peptides (Patho-topes Array) to detect NY-ESO-1 Ag-Sp T lymphocytes was specific for CD8 T lymphocytes and comprised 9 mers from SEQ ID NO 158 to SEQ ID NO 172.

The selection of peptides indicated in this example has been carried out by applying the same criteria used for the peptide selection in example 3.

1-73. (canceled)

74. A method for in vitro immuno-diagnosis of antigen-specific T lymphocytes based on the preparation of compositions, also called stimuli, able to stimulate the T lymphocytes; such compositions comprising at least one among the antigens in different forms selected in the group of: (a) raw protein extract, (b) purified or recombinant proteins, (c) synthetic peptides and combinations of (a), (b) and (c); such stimuli being identified as pathogen-specific when based on antigens originating from pathogens and vaccine-specific when based on antigens originating from strains used for making vaccines they; said method comprising the following steps:
i) isolation of peripheral blood mononuclear cells (PBMC) from a sample of human or animal venous blood;
ii) preparation of at least one stimulus selected between pathogen-specific and vaccine-specific stimuli,
iii) preparation of a negative control comprising cells cultivated in vitro in complete medium without stimuli and a positive control comprising cells cultivated in vitro in complete medium with an aspecific stimulus;
iv) stimulation of said T-lymphocytes with the vaccine-specific or the pathogen-specific stimulus in the presence of a costimulus;
v) incubation;
vi) selective staining by immunofluorescence;
vii) flow-cytometry acquisition and analysis;
viii) measurement and characterization of the immune response.

75. A method according to claim 74 where data evaluation and response are given by identifying a cut-off value for the specific response, set by common statistical methods as the average plus two times the standard deviation of the T cell response frequency obtained from a sample of healthy persons.

76. A method according to claim 74 where the aspecific stimulus is selected between phorbol myristic acetate and ionomycin.

77. A method according to claim 74 where PBMC are isolated from a sample of venous blood by centifugation on a density gradient.

78. A method according to claim 74 where the incubation in step v) is performed for one hour at 37° C. in a humidified CO₂ incubator, followed by an incubation of at least 3 hours in the presence of an inhibitor of the cellular secretion.

79. A method according to claim 74 wherein said selective staining of antigen-specific (Ag-Sp) T lymphocytes in step (vi) is performed by:
A) a monoclonal antibody against at least one T lymphocyte membrane antigens or subpopulation thereof;
B) a monoclonal antibody against a cytokine
C) a mixture of A) and B).

80. A method according to claim 79 wherein in item A) said T lymphocyte membrane antigens are chosen among: CD3, CD45, anti-CD4, CD8, CD25, CD27, CD38, CD45-RA, CD45-RO, CD69, CCR5, or CCR7.

81. A method according to claim 80 wherein said T lymphocyte membrane antigens are CD3 and CD45.

82. A method according to claim 79 wherein in item B) cytokines are selected from the group consisting of: interferon gamma, IL-2, IL-4, L-10,TNF-α,. MIP-1α, MIP-1β, RANTES, and corresponding mixtures.

83. A method according to claim 82 wherein said cytokine is interferon gamma.

84. A method according to claim 78 wherein said secretion inhibitor is selected between brefeldin-A and monensin.

85. A method according to claim 84 wherein said secretion inhibitor is brefeldin-A.

86. A method according to claim 74 wherein in step (iii) the co-stimulus is obtained by incubating the T-lymphocytes in the presence of an anti-CD28 and/or an anti-CD49d monoclonal antibody.

87. A method according to claim 74 to detect T-lymphocyte specific for infectious agents, tumor antigens, autoimmune antigens and allergenic agents.

88. A method according to claim 87 for in vitro diagnosis of infectious, autoimmune, allergic and neoplastic diseases.

89. A method according to claim 87 for detecting a resolution or a relapse of a pathology or for detecting the effectiveness of a chemotherapy or of a vaccination protocol.

90. A method according to claim 74 wherein the stimulus is selected in the group consisting of the peptides identified as SEQ ID NO 1 to SEQ ID NO 182.

91. A method according to claim 74 for the in vitro diagnosis of infectious diseases.

92. A method according to claim 74 for the in vitro diagnosis of biological threat agents infection.

93. A method according to claim 74 for the in vitro diagnosis of tumors.

94. A method according to claim 74 for the follow up of a chemotherapeutic treatment.

95. A method according to claim 74 for the in vitro diagnosis of in utero infections.

96. A method according to claim 74 for the in vitro diagnosis of post transplant infections.

97. Method according to claim 74 that is computer-made.

98. Software comprising the software paths that carry out the steps of the method claimed according to claim 74.

99. A method to design the peptides as in point (c) according to claim 74, said method comprising the following steps:
1) selection of a specific protein of a pathogen;
2) optionally definition of a “consensus sequence”, accounting for any possible strain or clade or subtype pathogen heterogeneity;
3) definition of the HLA Class I-binding peptides by SYFPEITHY (http://syfpeithi.bmi-heidelberg.com/) or BIMAS (http)://bimas.dcrt.nih.gov/molbio/hla_bind/);
4) selection of the peptides, with binding scores;
5) identification of immunodominant regions and of peptides which bind to at least two different HLA loci (HLA-A and -B, or HLA-A and -C, or HLA-B and -C), or preferably to all three loci (HLA-A and -B and -C);
6) identification of peptides of at least 9 aminoacid in length overlapping the immunodominant region;
7) selection of antigen-specific peptides by protein-protein BLAST (http://www.ncbi.nlm.nih.gov/blast/Blast.cgi);
8) design of a peptide mixture or composition.

100. A method according to claim 99 wherein pathogens are selected among: Variola (Ortho-Poxviruses), Anthrax (B. anthracis), Plague (Yersinia pestis), Tularemia (Francisella tularensis) and SARS (Coronavirus).

101. A method according to claim 99 wherein for variola and coronaviruses, proteins are selected from the core, from the surface/envelope and from regulatory proteins

102. A method according to claim 99 wherein for bacteria, proteins are selected among toxins associated to pathogenicity.

103. Method according to claim 99 that is computer-made.

104. Software comprising the software paths that carry out the steps of the method claimed according to claim 99.

105. Composition of peptides comprising at least one of following groups of peptides:
Ortho-Poxvirus peptides from sequence 1ID84 to 85, from sequence ID86 to 87, peptides from sequence ID88 to 90, peptides from sequences ID91 to 92, peptides sequence ID93, peptides from sequence ID94 to 95, peptides from sequence ID96 to 97, peptides from sequence ID98 to 99, peptides from sequence ID100 to 101, peptides from sequence ID102 to 103;
Anthrax (B.anthracis) peptides from sequence ID74 to 83;
SARS coronavirus: peptides from sequence ID44 to 59, peptides from sequence ID45 to 46 and from ID60 to 61, peptides from sequence ID47 to 48 and from ID62 to 63, peptides from sequence ID49 to 58 and from ID64 to 73, peptides from sequence ID45 to 46;Human non-SARS Coronavirus peptides from sequence ID173 to 177,
peptides from sequence ID178 to 182.

106. A composition for detecting specific T-lymphocyte activation comprising at least three peptides selected from the group consisting of peptides comprising at least 9 consecutive aminoacids comprised within anyone of the peptides from SEQ ID NO 1 to SEQ ID NO 182.

107. The composition according to claim 106 for immunodiagnosis of HIV infection comprising at least three HIV gag peptides selected from the group consisting of peptides comprising at least 9 consecutive aminoacids comprised within anyone of the following peptides: SEQ ID NO 1 to SEQ ID NO 20.

108. The composition according to claim 107 comprising at least three HIV gag peptides selected in the group consisting of: SEQ ID NO 1 to SEQ ID NO 20.

109. The composition according to claim 106 for immunodiagnosis of CMV infection comprising at least three peptides selected from the group consisting of peptides comprising at least 9 consecutive aminoacids comprised within anyone of the following peptides: SEQ ID NO 21 to SEQ ID NO 43.

110. The composition according to claim 109 comprising at least three peptides selected in the group consisting of SEQ ID NO 21 to SEQ ID NO 43.

111. The composition according to claim 106 for immunodiagnosis of SARS coronavirus infection comprising at least three peptides selected from the group consisting of peptides comprising at least 9 consecutive aminoacids comprised within anyone of the following peptides: SEQ ID NO 44 to SEQ ID NO 73.

112. The composition according to claim 111 comprising at least one peptide selected in the group consisting of SEQ ID NO 44 to SEQ ID NO 73.

113. The composition according to claim 112 comprising at least one of the SARS coronavirus E-protein derived peptide corresponding to SEQ ID NO 44 or to SEQ ID NO 59.

114. The composition according to claim 112 comprising at least three of the SARS coronavirus M-protein derived peptides corresponding to: SEQ ID NO 45, SEQ ID NO 46, SEQ ID NO ID60, SEQ ID NO 61.

115. The composition according to claim 112 comprising at least three of the SARS coronavirus N-protein derived peptides corresponding to: SEQ ID NO 47, SEQ ID NO 48, SEQ ID NO 62, SEQ ID NO 63.

116. The composition according to claim 112 comprising at least three peptides from each set of SARS coronavirus S-protein derived peptides wherein set 1) consists of SEQ ID NO 49 to SEQ ID NO 58 and set 2) consists of SEQ ID NO 64 to SEQ ID NO 73.

117. The composition according to claim 112 comprising at least one of the SARS coronavirus M-protein derived peptides corresponding to sequences: SEQ ID NO 45 and SEQ ID NO 46.

118. The composition according to claim 112 for immunodiagnosis of SARS infection specific for the Asian population (A-SARS) comprising at least three peptides from SEQ ID NO 44 to SEQ ID NO 58.

119. The composition according to claim 112 for immunodiagnosis of SARS infection specific for the Caucasian population (B-SARS) comprising at least three peptides selected from the group of: SEQ ID NO 59 to SEQ ID NO 73.

120. The composition according to claim 109 for immunodiagnosis of infectious diseases comprising at least two peptides selected from the group of peptides consisting of peptides comprising at least 9 consecutive aminoacids comprised within anyone of the following set of peptides: set 1) consisting of SEQ ID NO 1 to 20, set 2) consisting of: SEQ ID NO 21 to 43, set 3) consisting of: SEQ ID NO 44 to 73.

121. The composition according to claim 120 wherein said infectious diseases are AIDS, CMV and coronavirus (SARS) infections.

122. The composition according to claim 106 for immunodiagnosis of B.anthracis infection comprising at least three peptides selected from the group consisting of peptides comprising at least 9 consecutive aminoacids comprised within anyone of the following peptides: SEQ ID NO 74 to SEQ ID NO 83.

123. The composition according to claim 113 comprising at least three peptides selected from the group of: SEQ ID NO 74 to SEQ ID NO 83.

124. The composition according to claim 106 for immunodiagnosis of orthopoxviridae infection or vaccination comprising at least three peptides selected from the group consisting of peptides comprising at least 9 consecutive aminoacids comprised within anyone of the following peptides: SEQ ID NO 84 to SEQ ID NO 103.

125. The composition of claim 124 comprising at least three peptides selected from the group of: SEQ ID NO 84 to SEQ ID NO 103.

126. The composition according to claim 106 for immunodiagnosis of threat disease infections comprising at least two peptides selected from the group consisting of peptides comprising at least 9 consecutive aminoacids comprised within anyone of the following set of peptides: set 1 consisting of: SEQ ID NO 74 to 83, set 2 consisting of: SEQ ID NO 84 to 103.

127. A composition for immunodiagnosis of enteric infections comprising as immunostimulants comprising the following antigens: Shigella groups A, Al, B, C, C1, C2 antigens, Salmonella groups A, 0 antigens, Enterovirus 70 antigen lysate, HAV antigen lysate, HEV Hepatitis E Virus ORF2 antigen, Helicobacter pylori HPSa antigen, Clostridium difficile Toxin A antigen.

128. The composition according to claim 106 for detecting alpha-fetoprotein specific T-lymphocytes comprising at least three peptides selected from the group consisting of peptides comprising at least 9 consecutive aminoacids comprised within anyone of the following peptides: SEQ ID NO 104 to SEQ ID NO 122.

129. The composition according to claim 128 comprising at least three peptides selected in the group consisting of: SEQ ID NO 104 to SEQ ID NO 122.

130. The composition according to claim 106 for detecting of PSA specific T-lymphocytes comprising at least three peptides selected from the group consisting of peptides comprising at least 9 consecutive aminoacids comprised within anyone of the following peptides: SEQ ID NO 123 to SEQ ID NO 142.

131. The composition according to claim 130 comprising at least three peptides selected from the group of: SEQ ID NO 123 to SEQ ID NO 142.

132. The composition according to claim 106 for detecting MAGE-3 specific T-lymphocytes comprising comprising at least three peptides selected from the group consisting of peptides comprising at least 9 consecutive aminoacids comprised within anyone of the following peptides: SEQ ID NO 143 to SEQ ID NO 157.

133. The composition according to claim 132 comprising at least three peptides selected from the group of: SEQ ID NO 143 to SEQ ID NO 157.

134. The composition according to claim 106 for detecting NY-ESO-1 antigen specific T-lymphocytes comprising at least three peptides selected from the group consisting of peptides comprising at least 9 consecutive aminoacids comprised within anyone of the following peptides: SEQ ID NO 158 to SEQ ID NO 172.

135. The composition according to claim 134 comprising at least three peptides selected from the group of: SEQ ID NO 158 to SEQ ID NO 172.

136. The composition according to claim 106 for immunodiagnosis of tumors comprising at least two peptides selected from the group consisting of peptides comprising at least 9 consecutive aminoacids comprised within anyone of the following set of peptides: set 1 consisting of SEQ ID NO 104 to 122, set 2 consisting of SEQ ID NO 123 to 142, set 3 consisting of SEQ ID NO 143 to SEQ ID NO 157, set 4 consisting of SEQ ID NO 158 to SEQ ID NO 172.

137. The composition according to claim 136 wherein said tumors are selected from the group consisting of: melanoma, hepatocarcinomas, prostatic tumors, hesophageal tumors or tumors overexpressing at least one of the markers selected from: MAGE, PSA, NY-ESO-1, or AFP.

138. A kit to perform the immunodiagnostic method according to claim 74 comprising at least one of the compositions for detecting specific T-lymphocyte activation comprising at least three peptides selected from the group consisting of peptides comprising at least 9 consecutive aminoacids comprised within anyone of the peptides from SEQ ID NO 1 to SEQ ID NO 182, said kit further comprising, optionally in a freeze-dried form, at least one of the following components:
preparation of negative and positive control related to the specific antigenic composition
reagents, such as solution of washing and permeabilization,
reagents, such as mixtures of monoclonal antibodies,
pipettes and other laboratory material,
an instruction leaflet to perform the method according to claims 74.

139. The kit according to claim 138 for the combined immunodiagnosis of respiratory infection further comprising at least one of the purified proteins or antigen lysates selected in the group consisting of: Influenza A virus (H3N2) antigen lysate, Influenza A virus (H1N1) antigen, Influenza B virus (Hong Kong) antigen lysate, Influenza B virus (Victoria) antigen lysate, Influenza B virus (Tokio) antigen lysate, Influenza B virus (Qiengdao) antigen lysate, Influenza B virus (Lee) antigen lysate, Parainfluenza virus (group 1) antigen lysate, Parainfluenza virus (group 2) antigen lysate, Parainfluenza virus (group 3) antigen lysate, Parainfluenza virus (group 4) antigen lysate, Respiratory Syncytial Virus (RSV, ceppo A2) antigen lysate, SARS coronavirus recombinant protein E, SARS coronavirus recombinant protein M, SARS coronavirus recombinant protein Nucleocapsid aa. 1-49, SARS coronavirus recombinant protein Nucleocapsid aa. 192-220, echovirus 11 antigen lysate, Coxsackie B6 antigen lysate, Coxsackie A9 antigen lysate, Coxsackie A6 antigen lysate, adenovirus (Type 3) antigen lysate, adenovirus (type 6) antigen lysate, adenovirus (type 21) antigen lysate, Legionella pneumophila antigen (Trinity Biotech Plc, Wicklow, Ireland), Mycoplasma pneumoniae antigen lysate, Chlamidia pneumoniae, antigen lysate.

140. The kit according to claim 138 for the combined immunodiagnosis of enteric infections further comprising as immunostimulants the following antigens: Shigella groups A, A1, B, C, Cl, C2 antigens, Salmonella groups A, 0 antigens, Enterovirus 70 antigen lysate, HAV antigen lysate, HEV Hepatitis E Virus ORF2 antigen, Helicobacter pylon HPSa antigen, Clostridium difficile Toxin A antigen.

141. The kit according to claim 138 for the combined immunodiagnosis of sexually transmitted diseases further comprising at least one of the purified proteins or antigen lysates selected in the group consisting of: Treponema pallidum p15 recombinant antigen, Treponema pallidum p17 recombinant antigen, Treponema pallidum p45 recombinant antigen, Treponema pallidum TmpA recombinant antigen, HPV L1 capsid antigen recombinant protein, Candida albicans mixed antigen, HSV2 antigen lysate, HBV HBeAg recombinant antigen, HBV Core recombinant antigen, HBV HBsAg recombinant antigen, HIV-1 antigen lysate, HIV-2 antigen lysate, HIV-1 recombinant protein Gag, HIV-1 recombinant protein Nef, HIV-1 recombinant protein Env.

142. The kit according to claim 138 for the combined immunodiagnosis of in utero infections further comprising at least one of the purified proteins or antigen lysates selected in the group consisting of: Toxoplasma gondii lysate, Toxoplasma gondii Tachyzoites antigen, Rubella recombinant protein, CMV (AD 169) antigen lysate, CMV (AD 169) pp65 recombinant protein, CMV (AD169) pp150 recombinant, CMV (AD169) pp28 recombinant protein, CMV (AD169) pp38 recombinant protein, CMV (AD169) p50 recombinant protein, CMV (C194) gB recombinant protein,HSV-1 gDrecombinantprotein,HSV-1 gGrecombinantprotein, HSV-1 virallysate, VZV antigen lysate.

143. The kit according to claim 138 for the combined immunodiagnosis of post-transplant infections further comprising at least one of the purified proteins or antigen lysates selected in the group consisting of: CMV (AD169) antigen lysate, CMV (AD169) pp65 recombinant protein, CMV (AD169) pp150 recombinant, CMV (AD169) pp28 recombinant protein, CMV (AD169) pp38 recombinant protein, CMV (AD169) p50 recombinant protein, CMV (C194) gB recombinant protein, HSV-1 gD recombinant protein, HSV-1 gG recombinant protein, HSV-1 viral lysate, EBV (B95-8) antigen lysate.

144. The kit according to claim 138 for the combined immunodiagnosis of blood-borne infections further comprising at least one of the purified proteins or antigen lysates selected in the group consisting of: HIV-1 antigen lysate, HIV-2 antigen lysate, HIV-1 recombinant protein Gag, HIV-1 recombinant protein Nef, HIV-1 recombinant protein Env, HCV Core recombinant protein, HCV p22 nucleocapsid recombinant protein, HCV NS3 recombinant protein, HCV NS4 recombinant protein, HBV HBeAg recombinant antigen, HBV Core recombinant antigen, HBV HBsAg recombinant antigen, HDV delta antigen, recombinant, HGV antigen, recombinant, HHV-8 antigen lysate.

145. The kit according to claim 138 for the combined immunodiagnosis of threat-agent infections further comprising at least one of the purified proteins or antigen lysates selected in the group consisting of: Plague (Yersinia pestis) Capsular F1 antigen, Tularemia (Francisella tularensis) LPS antigen.
